(19) United States
(12) Patent Application Publication
(10) Pub. No.: US 2025/0281406 A1
(43) Pub. Date: Sep. 11, 2025

(54) COMPOUNDS, COMPOSITIONS AND METHODS FOR DELIVERY OF BIOLOGICALLY ACTIVE AGENTS

(71) Applicant: ROYAL MELBOURNE INSTITUTE OF TECHNOLOGY, Melbourne, Victoria (AU)

(72) Inventors: Nhiem TRAN, Melbourne, Victoria (AU); Sarigama RAJESH, Melbourne, Victoria (AU); Calum DRUMMOND, Melbourne, Victoria (AU); Jiali ZHAI, Melbourne, Victoria (AU)

(21) Appl. No.: 18/858,619

(22) PCT Filed: Apr. 21, 2023
(86) PCT No.: PCT/AU2023/050331

§ 371 Date: Oct. 21, 2024

(30) Foreign Application Priority Data
Apr. 22, 2022 (AU) 2022901069

(51) Int. Cl. A61K 9/1274; A61K 8/02; A61K 9/51; A61K 31/4745; A61K 47/22

(52) U.S. Cl. CPC A61K 9/1274; A61K 8/0295; A61K 9/5123; A61K 9/5146; A61K 31/4745; A61K 47/22

(57) ABSTRACT

The present disclosure relates to a lyotropic liquid crystalline lipid carrier which is useful for the delivery of active agents. The present disclosure also relates to cosmetic or pharmaceutical compositions comprising the lipid carrier, and methods of using the lipid carrier or compositions thereof to treat, prevent or diagnose diseases, disorders or conditions such as cancer or bacterial or fungal infections.

### CROSS-REFERENCE TO RELATED APPLICATIONS

The present application claims priority from Australian Provisional Patent Application No 2022901069 filed on 22 Apr. 2022, the contents of which are incorporated herein by reference in their entirety.

### TECHNICAL FIELD

The present disclosure relates to the field of nanomedicine. In particular, the present disclosure relates to lipid carriers or nanoparticles for delivery of biologically active agents. Disclosed herein are carriers, such as nanoparticles, comprising synthetic lipids, methods for preparation thereof, and uses of the nanoparticles for formulation and delivery of pharmaceutical or cosmetic agents.

### BACKGROUND

Any reference to background art herein is not to be construed as an admission that such art constitutes common general knowledge in Australia or elsewhere.

Pharmaceutical and cosmetic agents may be difficult to deliver for reasons such as poor solubility in aqueous medium, low cellular permeability, chemical and/or biological instability, rapid enzymatic and metabolic degradation, and elimination from the body, along with poor safety and tolerability. Many of such biologically active agents would be safer and more efficacious if they could be administered selectively. For example, chemotherapeutic drugs are often extremely toxic to both cancer cells and normal cells, and in most cases, there is no way to bias systemic delivery to the cancer cells to reduce off-target effects.

Different strategies exist for overcoming challenges associated with delivery of biologically active agents, such as new dosage forms, alternatives forms of agents (salts/complexes), prodrugs or other conjugates of drugs, or alternative routes of administration. Further examples include development of nanodelivery systems, such as liposomes, polymer micelles, microemulsions and self-emulsifying drug delivery systems, nanodispersions, inorganic nanoparticles, solid lipid nanoparticles, inclusion complexes, and nanodelivery systems based on chemical conjugation. Advances in the development of these technologies and their application to a wider range of biologically active agents are ongoing.

There is a need to provide additional carriers to deliver pharmaceutical and cosmetic agents, and other biologically active agents such as nucleic acids. There is a further need for the development of new carriers capable of preferential delivery of biologically active agents to a disease or infection site. Such carriers would be useful for disease therapy and diagnosis, and formulation of pharmaceuticals and personal care agents.

### SUMMARY

The present disclosure provides compounds, compositions and methods that are useful in any biological system where it may be desirable to deliver a material that is protected or otherwise separated from the system until specific conditions are present.

In particular, the present disclosure provides compounds, compositions and methods for improving the delivery of biologically active agents. Among other things, the present application provides compounds, compositions and methods for making and using novel delivery agents that are stable in circulation and which may undergo structural changes under appropriate physiological conditions (e.g., pH) which allow for control of the efficiency of delivery of biologically active agents.

The compounds, compositions, and methods provided by the present disclosure facilitate effective delivery of active agents by providing active agents encapsulated in lipid carriers that are stable in circulation at ambient pH and undergo structural changes under appropriate physiological conditions (e.g., decreased pH) which increase the release of the active agents in a local environment having those physiological conditions. Examples of such local environments having a decreased pH include tumours, sites affected by certain bacterial and fungal infections, and subcellular organelles.

Non-limiting examples of active agents that can be delivered using the compounds, compositions and methods of the present disclosure include both hydrophilic and lipophilic agents, including therapeutically or cosmetically active molecules, small molecule drugs, peptides, proteins, hormones, vitamins, antibodies, oligonucleotides, siRNAs, DNAs, mRNAs and imaging agents.

By exploiting the structural phase changes in the lipid carriers under appropriate physiological conditions (e.g., decreased pH), the release of encapsulated active agents from the lipid carriers can be controlled and engineered for targeted delivery of the active agents, improving efficacy and reducing off-target side effects of the active agents.

In a first aspect, there is provided a lyotropic liquid crystalline (LLC) lipid carrier comprising a structural lipid which is a lyotropic liquid crystal phase forming lipid, and an amino lipid having an amido-linker, the LLC lipid carrier adapted to undergo a mesophase transition upon exposure to a drop in pH.

In embodiments, the structural lipid is capable of forming a cubic mesophase structure. In embodiments, the structural lipid comprises a hydrophobic tail group selected from the group consisting of oleyl, linoleoyl, linolenoyl, phytanoyl, famesoyl or extended aliphatic hydrophobic, optionally selected from oleyl, linoleoyl, and phytanoyl. In embodiments, the structural lipid is monoolein (glycerol monooleate).

In embodiments, the amino lipid is present at a suitable wt % of the total lipid content of the LLC lipid carrier to provide for the mesophase transition upon exposure to a drop in pH. In embodiments, the amino lipid comprises about 5% to about 50 wt % of the total lipid content of the LLC lipid carrier.

In embodiments, the amino lipid has the structure of Formula (I):

Cyc-L-R (I),
wherein Cyc is a nitrogen heterocycle or heteroaryl;
L is an amido-linker; and
R is a C10 to C44 carbon chain.

In embodiments, the amino lipid has the structure of Formula (Ib):

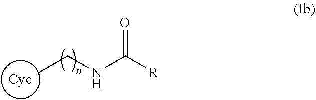

wherein Cyc is a 5- or 6-membered nitrogen heterocyclyl or heteroaryl, optionally selected from the group consisting of pyridyl, pyrimidinyl, piperidinyl, piperazinyl, morpholinyl, and pyrazinyl;
L is an amido-linker;
R is a C10 to C44 carbon chain, optionally a C12 to C24 alkyl or alkenyl, optionally interrupted by one or more heteroatoms; and
n is an integer from 1 and 6.

In embodiments, R is selected from the group consisting of oleyl, linoleoyl, linolenoyl, phytanoyl, and farnesoyl.

In embodiments, the amino lipid is selected from the group consisting of.

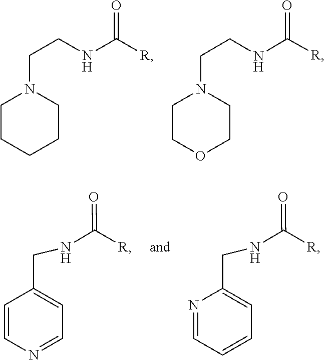

wherein, R is as defined above.

In embodiments, the LLC lipid carrier further comprises a stabilizer, optionally wherein the stabilizer is a stabilizing polymer, optionally a non-ionic triblock copolymer.

In embodiments, the stabilizer is present in an amount of about 10 wt % of the total lipid content of the LLC lipid carrier. the stabilizer is Pluronic F127 (Poloxamer 407) or Poloxamer 80.

In embodiments, the LLC lipid carrier is in the form of lyotropic liquid crystalline (LLC) nanoparticles.

In embodiments, the mesophase transition upon exposure to a drop in pH is a transition caused by a drop in pH below pH 7.

In embodiments, the mesophase transition upon exposure to a drop in pH is a transition caused by a drop in pH of 1 to 4 pH units.

In embodiments, the mesophase transition upon exposure to a drop in pH is a transition to a mesophase of lower interfacial curvature than the mesophase which the lipid carrier occupied prior to the pH drop.

In embodiments, the mesophase transition upon exposure to a drop in pH is a transition to a mesophase corresponding to lower CPP values.

In embodiments, the mesophase transition upon exposure to a drop in pH is a transition from a hexagonal phase to a cubic phase.

In embodiments, the mesophase transition upon exposure to a drop in pH is a transition from one mesophase to another which follows the mesophase transition order of L₂→H₂→Q₂→L₃. In embodiments, the mesophase transition is H₂→Q₂.

In embodiments, the LLC lipid carrier comprises a hexagonal lyotropic liquid crystalline phase structure at a pH of about pH 7 or higher, optionally about pH 7.4 or higher.

In embodiments, the LLC lipid carrier comprises a cubic lyotropic liquid crystalline phase structure at a pH of about pH 4.0-pH 7.0.

In embodiments, the LLC lipid carrier comprises a hexagonal lyotropic liquid crystalline phase structure at a pH of about pH 7 or higher, optionally about pH 7.4 or higher, and a cubic lyotropic liquid crystalline phase structure at a pH of about pH 4.0-pH 7.0.

In embodiments, the LLC lipid carrier further comprises an active agent, optionally wherein the active agent is pharmaceutically or cosmetically active. In embodiments, the active agent is selected from the group consisting of peptides, proteins, enzymes, small molecule drugs, and nucleic acids, optionally wherein the active agent is selected from the group consisting of radionuclides, imaging agents, polymers, antibiotics, fungicides, metal-containing nanoparticles, anti-inflammatory agents, anti-tumor agents, cardiovascular agents, anti-anxiety agents, hormones, growth factors, steroidal agents, gene expression modifiers, knockdown agents, siRNA, RNAi agents, mRNA, DNA, dicer substrates, miRNA, shRNA, antisense oligonucleotides, aptamers, and microbially derived toxins. In embodiments, the active agent is a topoisomerase I inhibitor, optionally selected from camptothecin, irinotecan, and SN-38.

In embodiments, the LLC lipid carrier has a water content of between about 20 to about 60% by weight.

In a second aspect, there is provided a lyotropic liquid crystalline (LLC) lipid nanoparticle composition which is a dispersion of the LLC lipid carrier of the first aspect in a polar medium.

In a third aspect, there is provided a cosmetic composition comprising the LLC lipid carrier of the first aspect, or the, or the LLC lipid nanoparticle composition of the second aspect, and a cosmetically acceptable carrier, diluent and/or excipient.

In a fourth aspect, there is provided a pharmaceutical composition comprising the LLC lipid carrier of the first aspect, or the LLC lipid nanoparticle composition of the second aspect, and a pharmaceutically acceptable carrier, diluent and/or excipient.

In embodiments, the cosmetic composition of the third aspect, or the pharmaceutical composition of the fourth aspect, is formulated as a composition for injection, for topical administration, or for subcutaneous administration.

In a fifth aspect, there is provided a method of delivering an active agent to a biological system including the step of administering to the biological system the LLC lipid carrier of the first aspect, the LLC lipid nanoparticle composition of the second aspect, or the composition of the third or fourth aspect.

In embodiments, the delivery of the active agent in the biological system is modified by a response of the LLC lipid carrier to a predefined pH range. In embodiments, the response comprises a mesophase structure transition of the LLC lipid carrier, optionally wherein the phase transition is a transition from a hexagonal phase structure to a cubic phase structure of the LLC lipid carrier. In embodiments, the mesophase structure transition occurs at a pH of about pH 7.0-pH 4.0, optionally about pH 7.0-pH 5.5. In embodiments, the response provides for a preferential release of the active agent at the predefined pH range in the biological system.

In a sixth aspect, there is provided a method of controlled release of an active agent comprising the steps of forming the LLC lipid carrier of the first aspect, the LLC lipid nanoparticle composition of the second aspect, or the composition of the third or fourth aspect, and administering the LLC lipid carrier, the LLC lipid nanoparticle composition, or the composition to a biological system comprising a target area having a predefined pH to thereby achieve a preferential release of the active agent at the target area.

In embodiments, the LLC lipid carrier undergoes a mesophase structure transition in response to the predefined pH. In embodiments, the mesophase structure transition is a transition from a hexagonal phase structure to a cubic phase structure of the LLC lipid carrier.

In a seventh aspect, there is provided a method of treatment or prevention of a disease, disorder or condition in a mammal, comprising administering to the mammal a therapeutically effective amount of the LLC lipid carrier of the first aspect, the LLC lipid nanoparticle composition of the second aspect, or the composition of the third or fourth aspect.

In an eighth aspect, there is provided a the LLC lipid carrier of the first aspect, the LLC lipid nanoparticle composition of the second aspect, or the composition of the third or fourth aspect, for use in the treatment or prevention of a disease, disorder or condition.

In a ninth aspect, there is provided a use of the LLC lipid carrier of the first aspect, the LLC lipid nanoparticle composition of the second aspect, or the composition of the third or fourth aspect, in the manufacture of a medicament for the treatment of a disease, disorder or condition.

In embodiments, the disease, disorder or condition is a cancer or a bacterial or fungal infection.

In a tenth aspect, there is provided a method of diagnosing a disease, disorder or condition in a mammal including the step of administering the LLC lipid carrier of the first aspect, the LLC lipid nanoparticle composition of the second aspect, or the composition of the third or fourth aspect, wherein the active agent is a labelled active agent, to the mammal or to a biological sample obtained from the mammal to facilitate diagnosis of the disease disorder or condition in the mammal.

In embodiments, the disease, disorder or condition is a cancer or a bacterial or fungal infection.

In an eleventh aspect, there is provided an amino lipid of Formula (Ib):

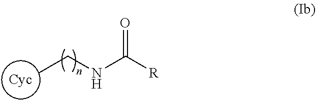

wherein Cyc is a 5- or 6-membered nitrogen heterocyclyl or heteroaryl, selected from the group consisting of piperidinyl, piperazinyl, pyrrolinyl, pyrrolidinyl, pyrazolidinyl, imidazolinyl, imidazolidinyl, pyrrolinyl, pyrazolinyl, thiazolidinyl, thiomorpholinyl, tetrahydropyridyl, dihydropyridyl, tetrahydropyrinidinyl, pyrimidinyl, pyridyl, pyrrolyl, imidazolyl, pyrazolyl, oxazolyl, oxadiazolyl, pyrazinyl, tetrazolyl, thiazolyl, isoxazolyl, isothiazolyl, isoxazolonyl, triazolyl, oxadiazolyl, thiadiazolyl, and pyridazinyl;
L is an amido-linker;
R is a C10 to C44 carbon chain, optionally a C12 to C24 alkyl or alkenyl, optionally interrupted by one or more heteroatoms; and
n is an integer from 1 and 6.

In embodiments, Cyc is selected from the group consisting of pyridyl, pyrimidinyl, piperidinyl, piperazinyl and pyrazinyl.

In embodiments, R is selected from the group consisting of oleyl, linoleoyl, linolenoyl, phytanoyl, and farnesoyl.

In embodiments, the amino lipid is selected from the group consisting of.

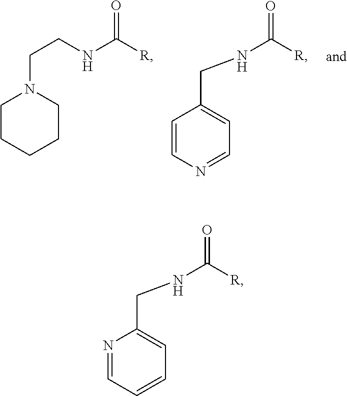

wherein, R is as defined above.

The various features and embodiments of the present invention, referred to in individual sections above apply, as appropriate, to other sections, mutatis mutandis. Consequently, features specified in one section may be combined with features specified in other sections as appropriate.

Further features and advantages of the present invention will become apparent from the following detailed description.

### BRIEF DESCRIPTION OF DRAWINGS

Whilst it will be appreciated that a variety of embodiments disclosed herein may be utilised, in the following, described herein are a number of examples with reference to the following drawings:

FIG. 1 is a schematic of pH dependent phase transition of lipid carriers described in the present application used for drug delivery in the treatment of tumours.

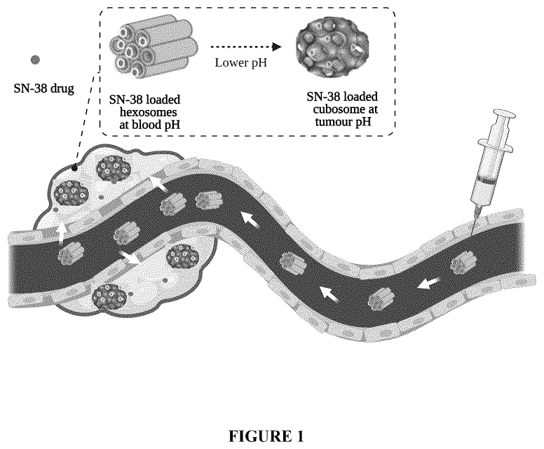

FIG. 2 shows chemical structure of exemplary synthesised amino lipids.

FIG. 3 is a series of SAXS derived partial lipid-water phase diagrams at 25° C. by adding increasing amounts of amino lipid to MO dispersed with F-127 across a pH range from 3.0 to 10. Highlighted boxes identify the key phase transition from hexagonal to cubic. A) Partial phase diagrams for nanoparticles obtained by incorporating OAPy-4, B) partial phase diagrams for nanoparticles with OAPy-2, C) partial phase diagrams for nanoparticles with OAMo-1, and D) partial phase diagrams for nanoparticles with OAPi-1. The amount of amino lipid added to the formulation is represented by RAL (also referred to as R_{MO} herein), which is defined as the wt/wt ratio of amino lipid to total lipid. N/D is a non-determined and non-identifiable phase. Identifiable phases include bicontinuous cubic (Im3m is primitive cubic, Pn3m is double diamond cubic, and Ia3d is gyroid cubic), sponge phase (L₃), inverse micelles (L₂), hexagonal (H₂), and lamellar (L_{α}).

FIG. 4 is a graphical representation of the effect of the synthesised amino lipids and pH on the mesophase structure of the MO nanoparticles.

FIG. 5 A) Representative one-dimensional SAXS profile for nanoparticles prepared by doping OAPy-4 to MO at R_{MO}=0.4. a) Results at 37° C. B) Results at room temperature.

FIG. 6 is a graphical representation of the particle size and polydispersity index (PDI) of the lipid nanoparticles loaded with SN-38. Measurements were averaged from triplicate, and the results were reported as a mean+/−standard deviation.

FIG. 7 is a SAXS partial phase diagrams of the lipid nanoparticles loaded with SN-38 at different wt % of SN-38 and the amino lipid. Highlighted boxes identify the key phase transition from hexagonal to cubic mesophase structure.

FIG. 8 is a graphical representation of the amount of drug loading determined for the lipid nanoparticles loaded with SN-38 at different wt % of SN-38 and the amino lipid. Measurements were averaged from triplicate, and the results were reported as a mean+/−standard deviation.

FIG. 9 is a graphical representation of the release of SN-38 drug from the lipid nanoparticle prepared using the amino lipid OAPy-4 (N-(pyridine-4-ylmethyl)oleamide). Nanoparticles were suspended in 1000 mL of release medium of PBS buffer pH 7 and pH 5. SN-38 loaded nanoparticles are hexosomes at neutral pH and cubosomes at pH 5. At predetermined time intervals (60 min, 120 min, 240 min and 420 min), 10 μL of the sample was withdrawn from the dialysis tube and diluted with 990 μL of HPLC solvent. The HPLC method quantitatively measured the SN-38 that remained in the dialysis tube. Experiments were performed in triplicate on three independently prepared SN-38 loaded formulations, and all values are expressed as mean±SD (n=3).

### DETAILED DESCRIPTION

Unless defined otherwise, all technical and scientific terms used herein have the same meaning as would be commonly understood by those of ordinary skill in the art to which this invention belongs.

All publications discussed and/or referenced herein are incorporated herein in their entirety, unless described otherwise.

Unless otherwise required by context, singular terms shall include pluralities and plural terms shall include the singular. Throughout this disclosure, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or groups of compositions of matter. Thus, as used herein, the singular forms “a” “an” and “the” include plural aspects unless the context clearly dictates otherwise. For example, reference to “a” includes a single as well as two or more; reference to “an” includes a single as well as two or more; reference to “the” includes a single as well as two or more and so forth.

Those skilled in the art will appreciate that the disclosure herein is susceptible to variations and modifications other than those specifically described. It is to be understood that the disclosure includes all such variations and modifications. The disclosure also includes all of the examples, steps, features, methods, compositions, formulations, and processes, referred to or indicated in this specification, individually or collectively, and any and all combinations or any two or more of said steps or features.

The term “and/or”, e.g., “X and/or Y” shall be understood to mean either “X and Y” or “X or Y” and shall be taken to provide explicit support for both meanings or for either meaning.

Unless otherwise indicated, the terms “first,” “second,” etc. are used herein merely as labels, and are not intended to impose ordinal, positional, or hierarchical requirements on the items to which these terms refer. Moreover, reference to a “second” item does not require or preclude the existence of lower-numbered item (e.g., a “first” item) and/or a higher-numbered item (e.g., a “third” item).

As used herein, the phrase “at least one of” or “one or more of” when used with a list of items, means different combinations of one or more of the listed items may be used and only one of the items in the list may be needed. The item may be a particular object, thing, or category. In other words, “at least one of” means any combination of items or number of items may be used from the list, but not all of the items in the list may be required. For example, “at least one of item A, item B, and item C” may mean item A; item A and item B; item B; item A, item B, and item C; or item B and item C. In some cases, “at least one of item A, item B, and item C” may mean, for example and without limitation, two of item A, one of item B, and ten of item C; four of item B and seven of item C; or some other suitable combination.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

Throughout the present specification, various aspects and components of the disclosure can be presented in a range format. The range format is included for convenience and should not be interpreted as an inflexible limitation on the scope of the present disclosure. Accordingly, the description of a range should be considered to have specifically disclosed all the possible sub-ranges as well as individual numerical values within that range, unless specifically indicated. For example, description of a range such as from 1 to 5 should be considered to have specifically disclosed sub-ranges such as from 1 to 3, from 1 to 4, from 1 to 5, from 2 to 4, from 2 to 5, from 3 to 5 etc., as well as individual and partial numbers within the recited range, for example, 1, 2, 3, 4, 5, 5.5 and 6, unless where integers are required or implicit from context. This applies regardless of the breadth of the disclosed range. Where specific values are required, these will be indicated in the specification.

In this patent specification, the terms ‘comprises’, ‘comprising’, ‘includes’, ‘including’, or similar terms are intended to mean a non-exclusive inclusion, such that a method or composition that comprises a list of elements does not include those elements solely, but may well include other elements not listed.

By “consisting of” is meant including, and limited to, whatever follows the phrase “consisting of” Thus, the phrase “consisting of” indicates that the listed elements are required or mandatory, and that no other elements may be present.

By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Herein, unless indicated otherwise, the term “about” encompasses a 10% tolerance in any value or values connected to the term.

Herein “weight %” may be abbreviated to as “wt %” or “wt. %”. “Pharmaceutical composition” or “pharmaceutical formulation” refers to a composition suitable for pharmaceutical use in a subject, including humans and mammals. A pharmaceutical composition comprises a pharmacologically effective amount of a composition used in the methods described herein and also comprises a pharmaceutically acceptable carrier. A pharmaceutical composition encompasses a composition comprising the active ingredient(s), and the inert ingredient(s), for example excipients, that make up the carrier, as well as any product which results, directly or indirectly, from combination, complexation or aggregation of any two or more of the ingredients, and/or from dissociation of one or more of the ingredients, and/or from other types of reactions or interactions of one or more of the ingredients. Accordingly, the pharmaceutical compositions of the present invention encompass any composition made by admixing a composition used in the methods described herein and a pharmaceutically acceptable carrier.

“Pharmaceutically acceptable carrier” or merely “carrier”, unless alternatively defined, refers to any of the standard pharmaceutical carriers, buffers, and excipients. The pharmaceutically acceptable carrier may be a saline solution, for example a phosphate buffered saline solution, or a 5% aqueous solution of dextrose. Further examples of a pharmaceutically acceptable carrier include but are not limited to emulsions, such as an oil/water or water/oil emulsion, and various types of wetting agents and/or adjuvants. Suitable pharmaceutical carriers and formulations are described in Remington's Pharmaceutical Sciences, 19th Ed. (Mack Publishing Co., Easton, 1995). Preferred pharmaceutical carriers depend upon the intended mode of administration of the active agent. Example modes of administration include enteral (e.g., oral) or parenteral (e.g., subcutaneous, intramuscular, intravenous or intraperitoneal injection; or topical, transdermal, or transmucosal administration).

The term “pharmaceutically acceptable salt”, as used herein, refers to salts of the active agent which are toxicologically safe for systemic or localised administration such as salts prepared from pharmaceutically acceptable non-toxic bases or acids including inorganic or organic bases and inorganic or organic acids. The pharmaceutically acceptable salts may be selected from the group including alkali and alkali earth, ammonium, aluminium, iron, amine, glucosamine, chloride, sulphate, sulphonate, bisulphate, nitrate, citrate, tartrate, bitarate, phosphate, carbonate, bicarbonate, malate, maleate, napsylate, fumarate, succinate, acetate, benzoate, terephthalate, palmoate, piperazine, pectinate and S-methyl methionine salts and the like.

As used herein, the term “cosmetic composition” refers to a composition used in cosmetics. Cosmetics are products that are intended to be rubbed, poured, sprinkled, or sprayed on, introduced into, or otherwise applied to the human body, for cleansing, beautifying, promoting attractiveness, or altering the appearance. Typically, cosmetic compositions do not provide a therapeutic effect and are not formulated as pharmaceuticals. However, in some situations, the cosmetic composition is incorporated into a pharmaceutical product to provide a cosmetic benefit (e.g., treats a skin or hair condition). The present disclosure also contemplates the use of cosmetic products on animals other than humans.

As used herein, “cosmetically acceptable” indicates that a particular component or excipient is generally regarded as safe and non-toxic at the levels employed.

As used herein the term “complex” is understood to mean a non-covalent physical interaction between two or more chemical entities.

The terms “nonlamellar lyotropic liquid crystalline phase” and “lyotropic liquid crystalline (LLC) lipid carrier”, as used herein, refer to a self-assembled nonlamellar liquid crystalline phase, formed from at least one amphiphile to give a two and/or three-dimensional mesophase structure which is capable of carrying an active agent. Dispersed nonlamellar lyotropic liquid crystalline phase particles are shown herein to result in pH-responsive structural behaviour. The term nonlamellar refers to the lyotropic liquid crystalline phase or lipid carrier or particle not being a liposome (or L_{α} phase) i.e. not presenting a planar structure, consisting of lipid bilayers separated by water, where the polar head groups of the amphiphilic molecules associate and are in contact with water directly, while the hydrophobic tails are away from water. Liquid crystalline phases, as described herein, are substances that exhibit a phase of matter that has properties between those of a conventional liquid, and those of a solid crystal. There are different types of liquid crystalline phases, which can be distinguished based on their different optical properties and other properties as are known in the art.

In embodiments, the LLC lipid carriers of the invention comprise only liquid crystals. That is, the LLC lipid carriers of the invention do not comprise any solid lipid component. The LLC lipid carriers of the invention are therefore not solid lipid nanocarriers (SLNs) or nanostructured lipid carriers (NLCs).

In embodiments, the terms “nonlamellar lyotropic liquid crystalline phase” or “lyotropic liquid crystalline phase” encompass only cubic, hexagonal and sponge morphologies. While the “sponge phase” or “sponge particles” (L₃) are recognised as not possessing long range order and demonstrating equivalent crystalline periodicity of the inverse bicontinuous cubic phase (Q₂), they are often considered as a “melted” Q₂ cubic phase and so are considered to be included as particles of the first or second aspect. Therefore, short range order sponge phases are explicitly considered to be within the scope of this term. In embodiments, the terms “nonlamellar lyotropic liquid crystalline phase” or “lyotropic liquid crystalline phase” may be used to include one or more phases selected from the group consisting of hexagonal (normal and reversed), cubic (normal discrete, reversed discrete, reversed bicontinuous—including primitive, gyroid and diamond—and reversed discontinuous), and other ‘intermediate phases’ including the ribbon, mesh, or non-cubic ‘sponge’ bicontinuous phases.

### Amino Lipid

The present disclosure provides, in a first aspect, a lyotropic liquid crystalline (LLC) lipid carrier comprising a structural lipid which is a lyotropic liquid crystal phase forming lipid, and an amino lipid having an amido-linker, the LLC lipid carrier adapted to undergo a mesophase transition upon exposure to a drop in pH.

In embodiments, the amino lipid has the structure of Formula (I):

Cyc-L-R (I),
wherein Cyc is a nitrogen heterocycle or heteroaryl;
L is an amido-linker; and
R is a C10 to C44 carbon chain.

The term “amino lipid”, as used herein, refers to amphiphilic lipid compounds comprising a functional group comprising a nitrogen atom, preferably a head group comprising a nitrogen atom. In embodiments, the lipid compounds comprise a basic ionizable functional group such as an amine or a nitrogen-containing aromatic ring. In embodiments, the amino lipids disclosed herein are ionisable and/or cationic lipids. In embodiments, the amino lipid provides pH sensitivity to the LLC lipid carrier disclosed herein. In any embodiments disclosed herein, the lipid may be referred to as an “amino lipid” based on the presence of at least one nitrogen atom.

In embodiments disclosed herein, the amino lipids generally comprise a polar, hydrophilic head group comprising a nitrogen heterocycle or heteroaryl, and a non-polar, hydrophobic tail group, comprising a C10 to C44 carbon chain. In embodiments, the amino lipid in the LLC lipid carrier disclosed herein may generally comprise one or more cationic and/or ionizable head-groups.

In embodiments, the nitrogen heterocycle or heteroaryl is a 5- or 6-membered nitrogen heterocyclyl or heteroaryl, such as for example piperidinyl, piperazinyl, pyrrolinyl, pyrrolidinyl, pyrazolidinyl, morpholinyl, imidazolinyl, imidazolidinyl, pyrrolinyl, pyrazolinyl, thiazolidinyl, thiomorpholinyl, tetrahydropyridyl, dihydropyridyl, tetrahydropyrinidinyl, pyrimidinyl, pyridyl, pyrrolyl, imidazolyl, pyrazolyl, oxazolyl, oxadiazolyl, pyrazinyl, tetrazolyl, thiazolyl, isoxazolyl, isothiazolyl, isoxazolonyl, triazolyl, oxadiazolyl, thiadiazolyl, or pyridazinyl group. In certain preferred embodiments, the nitrogen heterocycle or heteroaryl is selected from pyridyl, pyrimidinyl, piperidinyl, piperazinyl, morpholinyl, and pyrazinyl. Preferably, the nitrogen heterocycle or heteroaryl is pyridyl or piperidinyl.

In embodiments, the amino lipid in the LLC lipid carrier disclosed herein may comprise a cationic ionizable amino head-group to which is bound (e.g., covalently bound), via an amido-linker, a non-polar, hydrophobic tail group, comprising a C10 to C44 carbon chain.

As used herein, the term “amido-linker” refers to a linker comprising an amide group, such as an alkyl chain interrupted by an amide group.

In embodiments, the amido-linker comprises an optional carbon chain between the nitrogen heterocycle or heteroaryl and the amide group. In embodiments, the optional carbon chain comprises from 1 to 6 carbon atoms, or from 1 to 5 carbon atoms, or from 1 to 4 carbon atoms, or from 1 to 3 carbon atoms, or from 1 to 2 carbon atoms, or 1 carbon atom; preferably from 1 to 4 carbon atoms.

In embodiments, when present, the carbon chain is optionally interrupted by one or more heteroatoms. In embodiments, the heteroatoms for each interruption may be independently selected from O, N and S.

In embodiments, the non-polar, hydrophobic tail group comprises a C10 to C44 carbon chain. The carbon chain may be linear or branched alkyl, alkenyl or alkynyl chain. In certain embodiments, the tail group comprises a C10 to C36 alkyl, alkenyl or alkynyl chain, a C12 to C24 alkyl, alkenyl or alkynyl chain, preferably a C12 to C24 alkyl or alkenyl. In certain preferred embodiments, the tail group comprises a C12 to C18 alkyl, alkenyl or alkynyl chain, preferably a C12 to C18 alkyl or alkenyl chain, preferably a C12 to C18 alkenyl chain.

For example, the non-polar, hydrophobic tail group may comprise an oleyl, linoleoyl, linolenoyl, phytanoyl, and famesoyl group; preferably an oleyl or linoleoyl; preferably an oleyl group.

In some embodiments, the LLC lipid carrier disclosed herein comprises an amino lipid that has the structure of Formula (Ib):

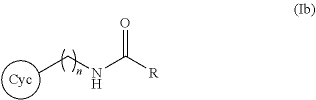

wherein Cyc is a nitrogen heterocycle or heteroaryl;
R is a C12 to C24 alkyl or alkenyl chain; and
n is an integer from 1 and 6, preferably from 1 to 4.

In embodiments, the Cyc group is a 6-membered nitrogen heterocycle or heteroaryl. In some embodiments, the Cyc is selected from the group consisting of pyridyl, pyrimidinyl, piperidinyl, piperazinyl, morpholinyl, thiomorpholinyl, tetrahydropyridyl, dihydropyridyl, tetrahydropyrinidinyl, pyrazinyl and pyridazinyl. Preferably, the Cyc is selected from the group consisting of pyridyl, pyrimidinyl, piperidinyl, piperazinyl, morpholinyl, and pyrazinyl.

In any embodiments disclosed herein, the R group of the amino lipid may be selected from those hydrophobic chains that follow the design rules for inverse bicontinuous cubic phases. The selection of the R group hydrophobic chain may be made on the basis of certain requirements which are understood in the art. For example, the hydrophobic chain may be chosen from those which promote a Type II lyotropic liquid crystalline phase at ambient and physiological temperatures. Parameters which may be appropriate for selection of an appropriate hydrophobic chain include: 1. The temperature for use should be above the chain melting temperature such that molten chains are present; and 2. There should be at least one cis unsaturated bond in a carbon chain of at least 14 carbons at a position at least mid-way along the backbone; or 3. The carbon backbone should contain at least 12 carbons of which 3 are secondary carbons with methyl branches; and 4. The molecular weight of the hydrophobic chain should be at least >200 amu.

In embodiments, the R group of the amino lipid is C12 to C24 alkenyl, preferably a C14 to C22 alkenyl.

In embodiments, the R group is selected from the group consisting of oleyl, linoleoyl, linolenoyl, phytanoyl, and farnesoyl. In some embodiments, the R group is oleyl. In some embodiments, R is:

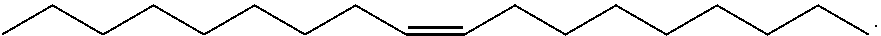 [SMILES: CCCCCCCC/C=C\CCCCCCCC]

In embodiments, the R group may be selected from those lipid tails known for use with polar head groups to form a cationic or ionisable cationic lipid. For example, the lipids below are known for formation of lipid nanoparticles (LNPs) for nucleic acid delivery. The lipid tails of these lipids, and those in this structural class, may be suitable for use as the R group as defined herein:

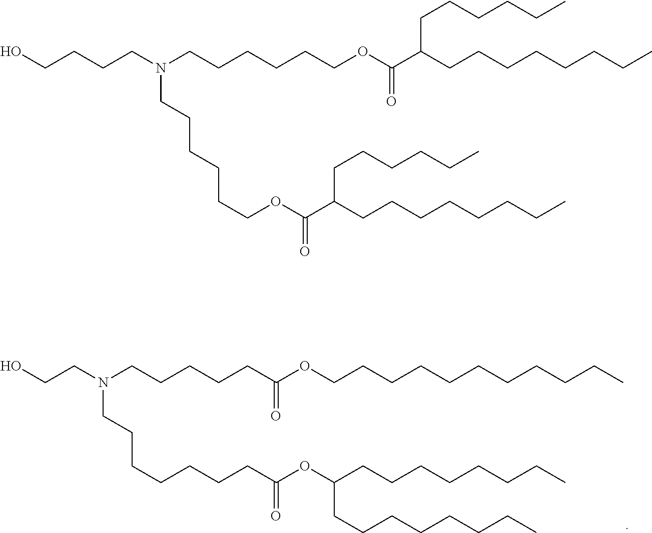 [SMILES: CCCCCCCCC(CCCCCC)C(=O)OCCCCCCN(CCCCO)CCCCCCOC(=O)C(CCCCCC)CCCCCCCC ; CCCCCCCCCCCOC(=O)CCCCCN(CCO)CCCCCCCC(=O)OC(CCCCCCCC)CCCCCCCC]

In embodiments, the amino lipid in the LLC lipid carrier disclosed herein is selected from the group consisting of:

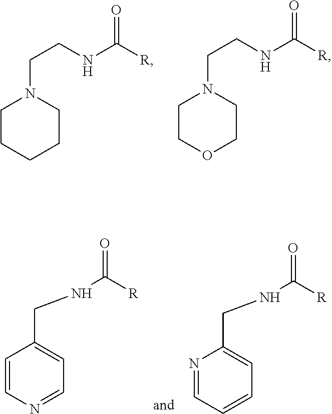

wherein, R is as defined above.

In some embodiments, the amino lipid is selected from the group consisting of

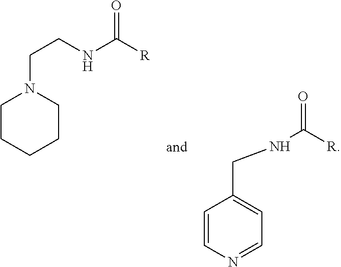

In another aspect, the present disclosure provides an amino lipid of Formula (Ib):

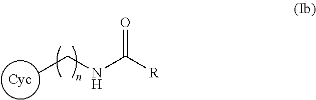

wherein Cyc is a 5- or 6-membered nitrogen heterocyclyl or heteroaryl, selected from the group consisting of piperidinyl, piperazinyl, pyrrolinyl, pyrrolidinyl, pyrazolidinyl, imidazolinyl, imidazolidinyl, pyrrolinyl, pyrazolinyl, thiazolidinyl, thiomorpholinyl, tetrahydropyridyl, dihydropyridyl, tetrahydropyrinidinyl, pyrimidinyl, pyridyl, pyrrolyl, imidazolyl, pyrazolyl, oxazolyl, oxadiazolyl, pyrazinyl, tetrazolyl, thiazolyl, isoxazolyl, isothiazolyl, isoxazolonyl, triazolyl, oxadiazolyl, thiadiazolyl, and pyridazinyl;
L is an amido-linker;
R is a C10 to C44 carbon chain, optionally a C12 to C24 alkyl or alkenyl, optionally interrupted by one or more heteroatoms; and
n is an integer from 1 and 6.

In embodiments, Cyc is selected from the group consisting of pyridyl, pyrimidinyl, piperidinyl, piperazinyl and pyrazinyl.

In embodiments, R is selected from the group consisting of oleyl, linoleoyl, linolenoyl, phytanoyl, and farnesoyl.

In embodiments, the amino lipid is selected from the group consisting of:

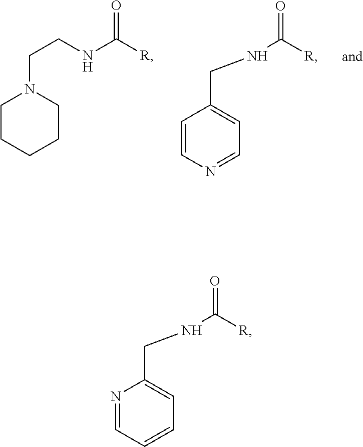

wherein R is as defined above.

In embodiments, the amino lipid is selected from the group consisting of:

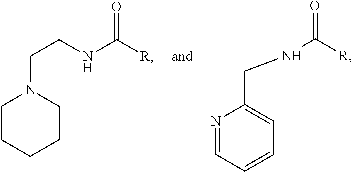

wherein R is oleyl.

The amino lipids described herein may be synthesized by any appropriate methods known to a person of skill in the relevant art.

For example, the amino lipids may be synthesized using an amide coupling reaction between the appropriate carboxylic acid or a derivative thereof carrying a hydrophobic tail group, and an amine carrying a hydrophilic head group. Alternatively, the amino lipids may be synthesized using an amide coupling reaction between the appropriate carboxylic acid or a derivative thereof carrying a hydrophilic head group, and an amine carrying a hydrophobic tail group. Conditions for amide coupling reactions are well-known in the art.

### Lyotropic Liquid Crystalline (LLC) Lipid Carrier

According to a first aspect, there is provided a lyotropic liquid crystalline (LLC) lipid carrier comprising a structural lipid which is a lyotropic liquid crystal phase forming lipid, and an amino lipid having an amido-linker, the LLC lipid carrier adapted to undergo a mesophase transition upon exposure to a drop in pH.

Lyotropic liquid crystalline (LLC) materials are generally composed of amphiphiles which self-assemble in a polar medium (e.g. aqueous solution or water) into various mesophase structures. Generally, the present disclosure relates to non-lamellar formed mesophases, which may consist of well-defined networks of aqueous channels and lipid bilayer membranes, such as for example bicontinuous cubic or inverse hexagonal mesophases. As used herein, the terms “mesophase”, “lyotropic phase”, “liquid crystalline phase”, “lyotropic liquid crystalline phase”, or just “phase” are used interchangeably. Non-lamellar mesophases can be applied as drug carriers either as bulk phases or as fabricated colloidal nanocarriers, e.g., cubosomes and hexosomes.

The terms “amphiphile”, “amphiphilic” and “amphiphilic lipid”, as used herein refer to compounds which comprise both a hydrophilic and a hydrophobic moiety. Typically, such compounds will have a hydrophilic head group and a hydrophobic tail. Suitable examples include fatty acids and a range of lipid molecules. In aqueous solutions, amphiphiles spontaneously form structures which hide the hydrophobic moieties but allow the hydrophilic head groups to be hydrated.

Herein, a “lyotropic liquid crystalline” material refers to a material having fluid properties similar to that of a viscous liquid and a degree of molecular order similar to a crystalline solid. The LLC lipid carrier of the present disclosure may have some or all components in the lyotropic liquid crystalline (LLC) phase. In embodiments, the LLC lipid carrier disclosed herein is at least 90% in the lyotropic liquid crystalline (LLC) phase. In some embodiments, the LLC lipid carrier disclosed herein does not comprise any solid crystal components.

Lyotropic phases are characterised in the art by the amount of curvature they exhibit. For example, where an amphiphilic monolayer bends towards the water phase, the lyotropic phase is called an inverse phase. In contrast, where a monolayer bends away from the water phase, the lyotropic phase is called a normal phase.

The LLC lipid carrier disclosed herein may be in the form of a LLC bulk phase. Bulk phase LLC materials are viscous, gel-like materials. The present disclosure relates, in general, to non-lamellar bulk self-assembled mesophases such as inverse bicontinuous cubic (Q₂), inverse hexagonal (H₂), or inverse micellar cubic (I₂) phases, which can be formed depending on the complex interplay between the molecular shape of the amphiphiles, the total free energy of the lipid-water system, and the environmental conditions.

Bulk phase LLC materials disclosed herein can be dispersed in an aqueous solution to produce LLC nanoparticles such as cubosomes and hexosomes. Accordingly, in embodiments, the LLC lipid carrier disclosed herein is in the form of LLC nanoparticles.

In contrast to the prior art lipid carriers, such as solid lipid nanoparticles (SLNs) or nanostructured lipid carriers (NLCs), the lipid carrier disclosed herein is a lyotropic liquid crystalline (LLC) lipid carrier, in which the amphiphiles retain fluid properties. As a result, the lyotropic liquid crystalline (LLC) lipid carrier disclosed herein advantageously can undergo a mesophase transition, such as a mesophase transition upon exposure to a change in pH (e.g., a drop in pH).

For example, the prior art SLNs described by Mhule et al. (Int. J. Pharm., 2018, 550, 149-159) are solid lipid nanoparticles comprising a crystallized amino lipid as the only lipid component, in addition to a surfactant and the drug vancomycin.

The LLC materials disclosed herein are typically formed in excess water condition, making them particularly suitable for drug delivery applications. For example, the LLC lipid carrier of the present disclosure may comprise water in an amount from about 0.1% to about 90% by weight, from about 0.1% to about 85% by weight, from about 0.10% to about 80% by weight, from about 0.10% to about 75% by weight, from about 0.10% to about 70% by weight, from about 0.10% to about 65% by weight, from about 0.10% to about 60% by weight, from about 0.10% to about 55% by weight, v, from about 0.1% to about 50% by weight, from about 0.1% to about 45% by weight, from about 0.1% to about 40% by weight, from about 0.1% to about 35% by weight, from about 0.1% to about 30% by weight, from about 0.1% to about 25% by weight, from about 0.1% to about 20% by weight, from about 0.1% to about 15% by weight, from about 0.1% to about 10% by weight, from about 0.1% to about 5% by weight, from about 0.1% to about 4% by weight, from about 0.1% to about 3% by weight, from about 0.1% to about 2% by weight, or from about 0.1% to about 1% by weight.

In embodiments, the LLC lipid carrier of the present disclosure comprises water in an amount of at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% by weight. In embodiments, the LLC lipid carrier of the present disclosure comprises water in an amount of at least 0.1%, at least 0.5%, at least 1%, at least 2.5%, at least 3%, at least 3.5%, at least 4%, at least 4.5%, or at least 5% by weight.

In embodiments, the LLC lipid carrier disclosed herein comprises water in an amount of about 1% to about 10% by weight, about 5% to about 15% by weight, about 10% to about 20% by weight, about 15% to about 25% by weight, about 20% to about 30% by weight, about 25% to about 35% by weight, about 30% to about 40% by weight, about 35% to about 45% by weight, about 40% to about 50% by weight, about 45% to about 55% by weight, about 50% to about 60% by weight, about 55% to about 65% by weight, about 60% to about 70% by weight, about 65% to about 75% by weight, or about 70% to about 80% by weight. In some embodiments, the LLC lipid carrier disclosed herein comprises water in an amount of about 10% to about 60% by weight, about 20% to about 60% by weight, about 30% to about 60% by weight, about 40% to about 60% by weight, or about 50% to about 60% by weight. In some embodiments, the LLC lipid carrier disclosed herein comprises water in an amount of about 40% to about 50% by weight.

The LLC lipid carrier disclosed herein is formed from at least one structural lipid which is a lyotropic liquid crystal phase forming lipid, in addition to the amino lipid having an amido-linker. In any embodiment herein, the at least one structural lipid is amphiphilic.

The at least one structural lipid may be selected from those amphiphilic lipids which are known in the art to form a lyotropic liquid crystal phase, particularly, cubic and/or mesophase structure, e.g. cubosomes and/or hexosomes. In embodiments, the structural lipid is capable of forming a cubic mesophase structure, e.g. cubosomes.

The selection of the appropriate at least one structural lipid may be made on the basis of certain requirements which are understood in the art. For example, the lipid(s) may be chosen from those which adopt a Type II lyotropic liquid crystalline phase at ambient and physiological temperatures. Parameters which may be appropriate for selection of an appropriate lipid include (i) on the hydrophobic component: 1. The temperature should be above the chain melting temperature such that molten chains are present; and 2. There should be at least one cis unsaturated bond in a carbon chain of at least 14 carbons at a position at least mid-way along the backbone; or 3. The carbon backbone should contain at least 12 carbons of which 3 are secondary carbons with methyl branches; and 4. The molecular weight of the hydrophobe should be at least >200 amu; and (ii) in relation to the head group: 5. The head group should contain at least three functional groups with minimum hydrophilicity (e.g. hydroxyl); 6. The head group should be able to form head group-water hydrogen bond networks; and 7. The head group area should be small relative to the hydrophobe footprint. By way of a guide, this is exemplified by the MO lipid used in the examples of the present disclosure as it: fulfils criteria 1, 2 and 4 for the hydrophobe; and fulfils criteria 5, 6 and 7 for the head group. It will be appreciated that many other lipids are available which fulfil these criteria appropriately and they may be selected on the basis of these criteria which are known, or easily ascertained, values.

Guidance on the selection of the at least one structural lipid may be found in one or more of the following publications which are each incorporated by reference herein in their entirety: (i) T. Kaasgaard and C. J. Drummond “Ordered 2D and 3D Nanostructured Amphiphile Self-Assembly Materials Stable in Excess Solvent” Phys. Chem. Chem. Phys. 2006, 8, 4957-4975. (ii) C. Fong, T. Le and C. J. Drummond “Lyotropic Liquid Crystal Engineering—Ordered Nanostructured Small Molecule Amphiphile Self-Assembly Materials by Design” Chem. Soc. Rev., 2012, 41, 1297-1322 DOI: 10.1039/clcs15148g; (iii) L. van 't Hag, S. L. Gras, C. E. Conn and C. J. Drummond “Lyotropic liquid crystal engineering moving beyond binary compositional space—Ordered nanostructured amphiphile self-assembly materials by design” Chem. Soc. Rev., 2017, 46, 2705-2731. DOI: 10.1039/c6cs00663a; and (iv) S. Sarkar, N. Tran, Md H. Rashid, T. C. Le, I. Yarovsky, C. E. Conn and C. J. Drummond “Toward cell membrane biomimetic lipidic cubic phases: a high-throughput exploration of lipid compositional space” ACS Applied Biomaterials, 2019, 2, 182-195. DOI: 10.1021/acsabm.8b00539.

Poly-hydroxyl (glycolipids) and polyethers (polyethylene oxides) form two of the largest categories of Type II forming head groups which may be of interest. Non-limiting examples of head group motifs include alcohols, fatty acids, monoacylglycerides, MAGs, 2-MAGs, glycerates, glyceryl ethers, ethylene oxides, amides, monoethanolamides, diethanolamides, serinolamides, methylpropanediolamides, ethylpropanediolamides, ureas, urea alcohols, biurets, biuret alcohols, ureides, endocannabinoids (anandamide, virodhamine, 2-glycerol, dopamine, 2-glycerol ether) and glycolipids. Examples include phospholipids such as DMPC and DMPE.

In embodiments, the at least one structural lipid may be selected from the group consisting of ethylene oxide-, monoacylglycerol-, glycolipid-, phosphatidylethanolamine-, and urea-based amphiphiles, and derivatives or analogues thereof.

Ethylene oxide amphiphiles may include C12(EO)2, C12(EO)4, C12(EO)5, and C12(EO)6 and dialkyl ethylene oxide amphiphiles. Monoacylglycerols may include monomyristolein, monoolein, monovaccenin and monoerucin. Amphiphiles resembling monoacylglycerols may be appropriate and include oleyl glycerate, phytanyl glycerate, glyceryl monooleyl ether, glyceryl phytanyl ether, phytantriol and monononadecenoin. Glycolipids with sugar moieties which may be appropriate including monosubstituted glycolipids: β-Mal3(Phyt)2, β-Glc(Phyt), β-Xyl(Phyt), β-Glc-(TMO)2, β-Mal2(Phyt)2 and β-Glc(Phyt)2; and disubstituted unbranched glycolipids: 1,2-diacyl-(β-D-glucopyranosyl)-sn-glycerols; 1,2-dialkyl-(β-D-glucopyranosyl)-sn-glycerols; 1,3-diacyl-(β-D-glucopyranosyl)-sn-glycerols; 1,3-dialkyl-(β-D-glucopyranosyl)-sn-glycerols. Phosphatidylethanolamine amphiphiles may include dioleoylphosphatidylcholine (DOPC) and dioleoylphosphatidylethanolamine (DOPE). Urea amphiphiles may include dodecylurea (DU), octadecylurea (ODU), oleylurea (OU), oleylbiuret (OBU), linoleylurea (LU), phytanylurea (PU), hexahydrofarnesyl-urea (HFU).

In embodiments, the at least one structural lipid may be selected from the group consisting of 1-monoolein, 2-monoolein, citrem, oleoyl lactate, oleamide, monoelaidin, linoleic acid, elaidic acid, monopalmitolein, monolinolein, phytantriol, diolein, triolein, dioleoyl-glycerol, didodecyldimethylammonium bromide, dioctadecyl (dimethyl) ammonium chloride (DOAC/DODMAC) or bromide (DODAB), 1,2-dioleoyl-sn-glycero-3-phosphoethanolamine (DOPE), 1,2-Dioleoyl-phosphatidylglycerol (DOPG), oleic acid, lysol-hydroxy-2-oleoyl-sn-glycero-3-phosphocholine, 1,2-dioleoyl-sn-glycero-3-dihexyl-phosphocholine, vitamin E tocopherol, vitamin E (tocopheryl) acetate, phytanoyl monoethanolamide, famesoyl monoethanolamide, oleoyl monoethanolamide, linoleoyl monoethanolamide and linolenoyl monoethanolamide.

Single-chain amphiphile lipids may be selected from the group consisting of saturated fatty acids C7-C16, oleic acid, elaidic acid, linoleic acid, sodium/gadolinium oleate, oleamide, 1-glyceryl monooleyl ether (GME), GMO, 2-MO, oleoyl lactate, citrem, Diglycerol monooleate (DGMO), Lyso (1-oleoyl)-phosphatidyl-choline, (Z)-Octadec-9-enylferrocene, N-Dodecyl-caprolactam (C12), Vitamin K1, ubiquinone-10 (coenzyme Q10), Vitamin E, Vitamin E acetate, Vitamin A palmitate, Alpha-tocopheryl PEO1000 succinate (vitamin E TPGS), PEG2000-MO, PEG-PT, PEOx-stearate (x=40-100), polysorbate 80.

Amphiphile lipids with multiple alkyl chains may be selected from the group consisting of didodecyldimethylammonium bromide (DDAB); Di(canola ethyl ester) dimethyl ammonium chloride (DEEDAC); Dioctadecyl (dimethyl) ammonium chloride (DOAC/DODMAC) or bromide (DODAB); diolein; Dioleoyl-glycerol (DOG), EDTA-bi-oleoyl; EDTA-bi-phytanyl; 1,2-Dioleoyl-3-trimethylammonium-propane (DOTAP); 1,2-Dioleoyl-phosphatidic acid (DOPA); 1,2-Dioleoyl-phosphatidylglycerol (DOPG), 1,2-Distearoyl-phophatidylglycerol (DSPG); 1,2-Dioleoyl-phosphatidylethanolamine (DOPE), 1,2-distearoyl-glycero-3-phosphoethanolamine (DSPE); 1,2-Dioleoyl-phosphatidylcholine (DOPC); 1-Palmitoyl-2-oleoyl-sn-glycero-3-phosphocholine (POPC); 1,2-Dioleoyl-sn-glycero-3-phosphoserine (DOPS); 1,2-Dipalmitoylphosphatidylserine (DPPS); DSPE-mPEG350, 750, 2000 (X=7, 16 or 45); DSPE-PEG2000, 3400, 5000; DMPE-mPEG550; (C18)2 DTPA (Gd), Cardiolipin, Cyclodextrin derivative (DCD-nC10).

In any embodiment herein, the at least one structural lipid may be a monoolein and/or phytantriol.

In any embodiment herein, the lipid carrier of the present disclosure may comprise MO or phytantriol in combination with one or more of cholesterol, DLPC, DSPC, DPPE, DPPS, DOPS, DPPC, DMPC, DMPS and DLPS.

Monoacylglycerols are known to form reversed phases over large regions of their phase diagrams, with monoolein being the most prominent. Formation of reversed phases is favoured because of the kink that is introduced by the cis-double bond. The longer acyl chain increases the hydrophobic chain volume and makes monoolein more wedge-shaped and shifted towards type 2 phases in the spectrum of mesophases. If the double bond is closer to the end of the lipid it diminishes its effect and makes it less wedge-shaped. Acyl chain extension is expected to drive the mesophase formation further towards the type 2 phases, and on this basis it is not surprising that the H₂-phase becomes the dominant phase with such a change.

In embodiments, wherein the LLC lipid carrier comprises at least two structural amphiphilic lipids, then at least one may be selected from a monoolein and phytantriol.

In such embodiments, the monoolein and/or phytantriol at least one structural lipid may, individually or in combination, be combined with one or more of triolein, vitamin E and DOPE. If it is desired to present a charge on the lipid carrier then these combinations may, themselves, further be combined with one or more cationic lipids selected from those which are well-known in the art and commercially available and including a wide variety of quaternary ammonium cationic compounds.

Certain of the at least one structural lipids may be chosen particularly because of their effect on the internal curvature of the final lipid particle, for example DOPE.

Representative cationic lipids may be selected from the following non-limiting examples: 3-β[4 N (1 N 8-diguanidinospermidine)-carbamoyl]cholesterol (BGSC); 3-β[N, N-diguanidinoethyl-aminoethane)-carbamoyl]cholesterol (BGTC); N, N 1, N 2, N 3 tetra-methyltetrapalmitylspermine (cellfectin); NtN′-butyl-N′-tetradecyl-3-tetradecyl-aminopropion-amidine (CLONfectin) Dimethyldioctadecyl ammonium bromide (DDAB); 1,2-myristoxypropyl-3-dimethyl-hydroxyethylammonium bromide (DMRIE); 2,3-dioleoyloxy-N-[2 (sperminecarboxamide) ethyl]-N, N-dimethyl-1-p-ropana (Nitrotrifluoroacetate) (DOSPA); 1,3-dioleoyloxy-2-(6-carboxyspermyl)-propylamide (DOSPER); 4-(2,3-bis-palmitoyloxy-propyl)-1-Methyl-1H-imidazole (DPIM); N, N, N′, N′-tetramethyl-N, N′-bis(2-hydroxyethyl)-2,3-dioleoyloxy-1,4-butane Diammonium iodide (Tfx-50); N-1-(2,3-dioleoyloxy) propyl-N, N, N-trimethylammonium chloride (DOTMA) or other N—(N, N-1-Dialkoxy)-alkyl-N, N, N-trisubstituted ammonium surfactants; trimethylammonium groups are double-stranded (DOT) via a butanol spacer arm 1,2-dioleoyl-3-(4′-trimethylammonio) butanol-sn-glycerol (DOBT) or cholesteryl (4′-trimethylammonia) butanoate (ChOTB) connected to a cholesteryl group (for ChOTB) DORI (DL-1,2-dioleoyl-3-dimethylaminopropyl-B-hydroxyethylammonium) or DORIE (DL-1,2-O-dioleoyl-3-dimethylaminopropyl) as disclosed in WO 93/03709-B-hydroxyethylammonium) (DORIE) or analogs thereof; 1,2-dioleoyl-3-succinyl-sn-glycerol choline ester (DOSC); tetraoctylammonium bromide (TOAB) as a cationic phase transfer agent; cholesteryl hemisuccinate ester (ChOSC); Midonglycylspermine (DOGS) and dipalmitoylphosphatidylethanol amylspermine (DPPES) or cationic lipids disclosed in U.S. Pat. No. 5,283,185, cholesteryl-30-carboxy-amido-ethylenetrimethylammonium chloride, 1-dimethyl Amino-3-trimethylammonio-DL-2-propyl-cholesterylcarboxylate iodide, cholesteryl-3-O-carboxyamidoethyleneamine, cholesteryl-3-β-oxysuccinamide-ethylenetrimethylammonium iodide, 1-Dimethylamino-3-trimethylammonio-DL-2-propyl-cholesteryl-3-β-oxysuccinate iodide, 2-(2-trimethylammonio)-ethylmethylaminoethyl-choles Teryl-3-β-oxysuccinate iodide, 3-β-N—(N′, N′-dimethylaminoethane) carbamoylcholesterol (DC-chol), and 3-β-N-(polyethyleneimine)-carbamoyl Cholesterol; O, O-Dimyristyl-N-lysyl-aspartate (DMKE); O, O-Dimyristyl-N-lysyl-glutamate (DMKD): 1,2-Dimyristyloxypropyl-3-dimethyl-hydroxyethylammonium bromide (DMRIE); 1,2-dilauroyl-sn-glycero-3-ethylphosphocholine (DLEPC); 1,2-dimyristyl-sn-glycero-3-ethylphosphocholine (DMEPC); 1,2-dioleoyl-sn-Glycero-3-ethylphosphocholine (DOEPC); 1,2-dipal Toyl-sn-glycero-3-ethylphosphocholine (DPEPC); 1,2-distearoyl-sn-glycero-3-ethylphosphocholine (DSEPC); 1,2-dioleoyl-3-trimethylammoniumpropane (DOTAP); Dioleoyl dimethylaminopropane (DODAP); 1,2-palmitoyl-3-trimethylammoniumpropane (DPTAP); 1,2-distearoyl-3-trimethylammoniumpropane (DSTAP); 1,2-myristoyl-3-trimethyl Ammonium propane (DMTAP); and sodium dodecyl sulfate (SDS).

In embodiments, particularly preferred cationic lipids are DOTAP and/or DODAB and/or tetraoctylammonium bromide (TOAB).

In embodiments the lipid carrier may comprise a fatty acid in addition to the at least one structural lipid such as, for example, oleic acid.

In further embodiments, suitable amphiphilic lipids include but are not limited to lipid compounds comprises a hydrophobic tail group selected from the group consisting of oleyl, linoleoyl, linolenoyl, phytanoyl, farnesoyl or extended aliphatic hydrophobic tail. In certain embodiments, the structural lipid compounds comprise an oleyl, linoleoyl, or phytanoyl hydrophobic tail, preferably an oleyl tail.

Generally, any amphiphilic lipid typically used to form cubic phase nanoparticles can be used as the structural lipid.

In embodiments, the one or more structural amphiphilic lipids may be selected from the group consisting of monoolein (also known as glycerol monooleate), citrem, oleoyl lactate, oleamide, monoelaidin, linoleic acid, elaidic acid, monopalmitolein, monolinolein, phytantriol, diolein, triolein, dioleoyl-glycerol, didodecyldimethylammonium bromide, dioctadecyl (dimethyl) ammonium chloride (DOAC/DODMAC) or bromide (DODAB), 1,2-dioleoyl-sn-glycero-3-phosphoethanolamine (DOPE), 1,2-Dioleoyl-phosphatidylglycerol (DOPG), oleic acid, lysol-hydroxy-2-oleoyl-sn-glycero-3-phosphocholine, 1,2-dioleoyl-sn-glycero-3-dihexyl-phosphocholine, vitamin E tocopherol, vitamin E (tocopheryl) acetate, phytanoyl monoethanolamide, farnesoyl monoethanolamide, oleoyl monoethanolamide, linoleoyl monoethanolamide and linolenoyl monoethanolamide.

In preferred embodiments, the structural amphiphilic lipid is monoolein.

In embodiments, the LLC lipid carrier is a nonlamellar lyotropic liquid crystalline phase, such as a self-assembled nonlamellar lyotropic liquid crystalline phase. In embodiments, the nonlamellar lyotropic liquid crystalline phase is formed by the self-assembly of the one or more amphiphilic lipids. It will be understood that appropriate amphiphilic lipids will self-assemble when in the presence of an aqueous solution, such as water or an aqueous buffer solution, to form a lyotropic liquid crystalline structure displaying a nonlamellar mesophase. In embodiments, the nonlamellar lyotropic liquid crystalline phase is a mesophase having inverse bicontinuous cubic (Q₂), inverse hexagonal (H₂), or inverse micellar cubic (I₂) structure.

The bulk lyotropic liquid crystalline phase can be dispersed to form nanoparticles. When dispersed in an aqueous solution, these nanoparticles retain their internal nanostructures and are called cubosomes, hexosomes, and micellar cubosomes.

Accordingly, in some embodiments, the LLC lipid carrier disclosed herein is in the form of a nanoparticle, such as a lipid nanoparticle (LNP). Such nanoparticles may be referred to as lyotropic liquid crystalline (LLC) lipid nanoparticles or lyotropic liquid crystalline lipid nanoparticles (LCNPs). The terms “nanoparticles”, “LLC lipid nanoparticles”, “LNP” and “LCNP” are used interchangeably herein.

The nanoparticles may be formed by dispersal of the bulk nonlamellar lyotropic liquid crystalline phase into colloidal systems. Upon dispersal, for example in an aqueous solution, nanoparticles such as cubosomes, hexosomes, and micellar cubosomes are produced, for example.

The LLC lipid carriers of the invention may preferably comprise at least one stabilizer selected from those known in the art. A suitable stabilizer may aid in the dispersal of the nonlamellar lyotropic liquid crystalline phase, and/or may aid to retain the nanostructure of the nanoparticles.

In embodiments, the stabilizer may be a Poloxamer or a surfactant or a PEGylated lipid stabilizer, or a modified version of these.

In embodiments, the stabilizer is selected from a PEG-PPO-PEG triblock copolymer and a non-ionic block copolymer surfactant and a PEO co-polymerised with a charged moiety. Poloxamer 407 and Pluronic 127 may be suitable examples of a stabilizing agent and may be incorporated into any of the embodiments of the first or second aspect described herein. PEO co-polymerised with (3-Acrylamidopropyl)trimethylammonium chloride, or a similar charge-carrying moiety, may also be appropriate. PEGylated lipid stabilizers are also appropriate including but not limited to PEG2000-MO, PEG-PT, DSPE-PEG(2000) Amine, 18:0 PEG2000 PE, and DSPE-PEG(5000) Amine. Many other such stabilizers are known in the art.

The use of a stabilizer is preferable and while the nature of the stabilizer may be selected based on the nature of the lipids, the selection and understanding of the compatibility of these components can be based upon information known in the art.

The many steric stabilizers which have been reported to date can be divided into four groups: (i) amphiphilic block copolymers (i.e. Poloxamer™), (ii) PEGylated lipids (iii) customized lipid-copolymers and (iv) alternative steric stabilizers (e.g., bile salts, proteins). Ideally the stabilizer selected will prevent aggregation of the particles by providing an electrostatic or, more commonly, steric barrier between approaching particles. Stabilizers which may function optimally in the lipid particles of the present disclosure share similar properties including (i) they are generally highly hydrophilic with a high HLB (hydrophilic-lipophilic balance) value due to an asymmetric amphiphilic polymer structure with a larger hydrophilic domain. It is important that the hydrophilic part of the molecule is not surrounded by hydrophobic regions. A high HLB may be achieved via use of longer PEG chains or multiple PEG chains; (ii) presence of hydrogen bond acceptors and absence of hydrogen bond donors; and (iii) electrically neutral. A person of skill in the art can select the appropriate stabilizer on this basis. Further, the following journal articles address key aspects of stabilizers which may be appropriate for use with the lipid carriers of the present disclosure and are hereby incorporated by reference in their entirety: (i) J. Y. T. Chong, X. Mulet, B. J. Boyd and C. J. Drummond; “Steric Stabilizers for Cubic Phase Lyotropic Liquid Crystal Nanodispersions (Cubosomes)” in “Advances in Planar Lipid Bilayers and Liposomes”, Vol 21, Chp 5, (2015) p. 131-187, ISSN 1554-4516, Elsevier; (ii) J. Zhai, B. Fan, S. H. Thang, C. J. Drummond “Novel amphiphilic block copolymers for the formation of stimuli-responsive non-lamellar lipid nanoparticles” Molecules, 2021, 26, 3648-3664. DOI: 10.3390/molecules26123648; (iii) J. Zhai, R. Suryadinata, B. Luan, N. Tran, T. M. Hinton, J. Ratcliffe, X. Hao and C. J. Drummond “Amphiphilic brush polymers produced by the RAFT polymerisation method stabilise and reduce the cell toxicity of lipid lyotropic liquid crystalline nanoparticles” Faraday Discussions, 2016, 191, 545-563. DOI: 10.1039/C6FD00039H; Faraday Discussion 191 on Nanoparticles with Morphological and Functional Anisotropy; (iv) J. Zhai, T. J. Hinton, L. J. Waddington, C. Fong, N. Tran, X. Mulet, C. J Drummond and B. W. Muir “Lipid-PEG Conjugates Sterically Stabilise and Reduce the Toxicity of Phytantriol-Based Lyotropic Liquid Crystalline Nanoparticles” Langmuir, 2015, 31, 10871-10880. DOI: 10.1021/acs.langmuir.5b02797; (v) J. Y. T. Chong, X. Mulet, D. Keddie, L. J. Waddington, S. T. Mudie, B. J. Boyd and C. J. Drummond “Novel Steric Stabilisers for Lyotropic Liquid Crystalline Nanoparticles: Pegylated Phytanyl Copolymers” Langmuir, 2015, 31, 2615-2629. DOI: 10.1021/la501471z; (vi) J. Y. T. Chong, X. Mulet, A. Postma, D. J. Keddie, L. J. Waddington, B. J. Boyd and C. J. Drummond “Novel RAFT Amphiphile Brush Copolymer Steric Stabilisers for Cubosomes: Poly(octadecyl acrylate)-block-poly(polyethylene glycol methyl ether acrylate)” Soft Matter, 2014, 10, 6666-6676. DOI: 10.1039/C4SM01064G; (vii) A. Tilley, C. J. Drummond and B. J. Boyd “Disposition and Association of the Steric Stabiliser Pluronic F127 in Lyotropic Liquid Crystalline Nanostructured Particle Dispersions” J. Colloid and Interface Science, 2013, 392, 288-296. DOI: 10.1016/j.jcis.2012.09.051 (viii) J. Y. T. Chong, X. Mulet, L. J. Waddington, B. J. Boyd and C. J. Drummond “High Throughput Discovery of Novel Steric Stabilisers for Cubic Lyotropic Liquid Crystal Nanoparticle Dispersions” Langmuir, 2012, 28, 9223-9232. DOI: 10.1021/la301874v; (ix) J. Y. T. Chong, X. Mulet, L. J. Waddington, B. J. Boyd and C. J. Drummond “Steric Stabilisation of Cubic Lyotropic Liquid Crystalline Nanoparticles: High Throughput Evaluation of Triblock Polyethylene Oxide-Polypropylene Oxide-Polyethylene Oxide Copolymers.” Soft Matter, 2011, 7, 4768-4777. DOI: 10.1039/clsm05181d.

The stabilizer may be present during formation of the LLC lipid nanoparticles disclosed herein.

Certain preferred stabilizers include stabilizing polymers, such as non-ionic triblock copolymers. In certain embodiments, the stabilizer is Pluronic F127 (also known as Poloxamer 407) or Poloxamer 80; preferably Pluronic F127.

The LLC lipid carrier disclosed herein may be prepared by mixing the amino lipid disclosed herein and the structural lipid. For example, the amino lipid and the structural lipid may be admixed as solutions in an organic solvent, followed by evaporation of the solvent. Preferably, at least stabilizer is also mixed with these lipid components.

A mixture of two or more structural lipids may be used as the structural lipid. Equally, two or more amino lipids may be used to form the lipid carrier of the present disclosure.

Various weight ratios of the amino lipid and the structural lipid may be used. These can be useful in determining the precise pH at which the desired structural transition occurs. The preferred weight ratio of the amino lipid to the structural lipid or total lipid within the lipid carrier can simply be determined by trialling multiple ratios and determining that which is optimal for the pH environment under consideration. Such an approach is set out in the experimental section. Therefore, in embodiments, the weight ratio of the amino lipid to the structural lipid or total lipid within the lipid carrier may be that which results in a mesophase transition upon a drop in pH to a previously determined pH value.

In embodiments, the structural lipid may make up the bulk of the lipid carrier, or be used as an equal proportion to the amino lipid. In embodiments, the ratio of the structural lipid to the lipid having an amido-linker, such as the amino lipid, is 95:5, 90:10, 85:15, 80:20, 75:25, 70:30, 60:40, 50:50, 40:60, 30:70, 20:80, 10:90, 5:95 wt %; preferably 95:5, 90:10, 85:15, 80:20, 75:25, 70:30, 60:40, 50:50, 40:60, 30:70 wt %; preferably 95:5, 90:10, 85:15, 80:20, 75:25, 70:30, 60:40, 50:50 wt %. In embodiments, the corresponding weight fraction of the amino lipid is 0.05, 0.1, 0.15, 0.2, 0.25, 0.3, 0.4, 0.5, 0.6, 0.7, 0.8, 0.9, 0.95; preferably 0.05, 0.1, 0.15, 0.2, 0.25, 0.3, 0.4 and 0.5, 0.6, 0.7; preferably 0.05, 0.1, 0.15, 0.2, 0.25, 0.3, 0.4 and 0.5.

In embodiments, the amino lipid having an amido-linker is present at a weight fraction with the total amount of lipid in the lipid carrier of between 0.1 to 0.7.

In embodiments, the amino lipid having an amido-linker is present at a weight fraction with the total amount of lipid in the lipid carrier of between 0.1 to 0.6, or 0.2 to 0.6, or 0.1 to 0.55 or 0.2 to 0.55.

In certain preferred embodiments, the weight fraction of the amino lipid is between 0.2 and 0.6, preferably between 0.3 and 0.5, preferably between 0.4 and 0.5.

In any embodiments described herein, the amino lipid may be present at a suitable wt % of the total lipid content of the LLC lipid carrier to provide for the mesophase transition upon exposure to a drop in pH. For example, the amino lipid may comprise about 5 wt % to about 50 wt % of the total lipid content of the LLC lipid carrier disclosed herein. The LLC lipid carrier may comprise the amino lipid in an amount of greater than about 5%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, or greater than about 80% of the total lipid content of the LLC lipid carrier. In embodiments, the amino lipid may comprise about 0.5 wt % to about 50 wt % of the total lipid content of the LLC lipid carrier disclosed herein, for example about 0.5 wt % to about 40 wt %, about 1 wt % to about 35 wt %, about 1.5 wt % to about 30 wt %, about 2 wt % to about 25 wt %, about 2.5 wt % to about 20 wt %, about 3 wt % to about 15 wt %, about 3.5 wt % to about 10 wt %, or about 4 wt % to 9 wt %, of the total lipid content. In an embodiment, the amino lipid comprises about 40 wt % of the total lipid content of the LLC lipid carrier disclosed herein.

To prepare the LLC lipid nanoparticles disclosed herein, the mixture of the amino lipid and the structural lipid may be dispersed in an aqueous solution. For example, the aqueous solution may be water, a buffer, or another aqueous solution. The aqueous solution may also contain the stabilizer. Suitable stabilizers include the stabilizers described above. In embodiments, the stabilizer may be a Poloxamer or a surfactant or a PEGylated lipid stabilizer, or a modified version of these. Preferred stabilizers include but are not limited to Pluronic F127 or Poloxamer 80.

The LLC lipid nanoparticles of the present disclosure may comprise water in n amount from about 0.1% to about 90% by weight, from about 0.1% to about 85% by weight, from about 0.1% to about 80% by weight, from about 0.1% to about 75% by weight, from about 0.1% to about 70% by weight, from about 0.1% to about 65% by weight, from about 0.10% to about 60% by weight, from about 0.10% to about 55% by weight, v, from about 0.1% to about 50% by weight, from about 0.1% to about 45% by weight, from about 0.10% to about 40% by weight, from about 0.10% to about 35% by weight, from about 0.10% to about 30% by weight, from about 0.10% to about 25% by weight, from about 0.10% to about 20% by weight, from about 0.10% to about 15% by weight, from about 0.10% to about 10% by weight, from about 0.10% to about 5% by weight, from about 0.1% to about 4% by weight, from about 0.1% to about 3% by weight, from about 0.10% to about 2% by weight, or from about 0.1% to about 1% by weight.

In embodiments, the LLC lipid nanoparticles disclosed herein comprise water in an amount of at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% by weight. In embodiments, the LLC lipid nanoparticles of the present disclosure comprise water in an amount of at least 0.1%, at least 0.5%, at least 1%, at least 2.5%, at least 3%, at least 3.5%, at least 4%, at least 4.5%, or at least 5% by weight.

In embodiments, the LLC lipid nanoparticles disclosed herein comprise water in an amount of about 1% to about 10% by weight, about 5% to about 15% by weight, about 10% to about 20% by weight, about 15% to about 25% by weight, about 20% to about 30% by weight, about 25% to about 35% by weight, about 30% to about 40% by weight, about 35% to about 45% by weight, about 40% to about 50% by weight, about 45% to about 55% by weight, about 50% to about 60% by weight, about 55% to about 65% by weight, about 60% to about 70% by weight, about 65% to about 75% by weight, or about 70% to about 80% by weight. In some embodiments, the LLC lipid nanoparticles disclosed herein comprise water in an amount of about 10% to about 60% by weight, about 20% to about 60% by weight, about 30% to about 60% by weight, about 40% to about 60% by weight, or about 50% to about 60% by weight. In some embodiments, the LLC lipid nanoparticles disclosed herein comprise water in an amount of about 40% to about 50% by weight.

The LLC lipid nanoparticles of the present disclosure may be provided as a nanoparticle dispersion in a polar medium, such as an aqueous medium. Accordingly, in a second aspect, there is provided a lyotropic liquid crystalline (LLC) lipid nanoparticle composition which is a dispersion of the LLC lipid carrier disclosed herein in a polar medium, such as an aqueous dispersion of the LLC lipid carrier disclosed herein. The term “polar medium” is to be understood to mean polar media including but not limited to water, glycerol, propylene glycol, propylene carbonate, methanol, ethanol, glycofurol and the like, and solutions based on these liquids, and mixtures thereof. For example, the polar medium could be water or an aqueous buffer. Generally, the LLC lipid nanoparticle compositions disclosed herein may include a solvent or dispersion medium comprising but not limited to water, ethanol, polyol (e.g., glycerol, propylene glycol, liquid polyethylene glycol, etc.), lipids (e.g., triglycerides, vegetable oils, and injectable organic esters such as ethyl oleate), and combinations thereof. In some cases, it will be preferable to include isotonic agents, such as, for example, sugars, sodium chloride or combinations thereof.

In some embodiments, the LLC lipid nanoparticle compositions disclosed herein will be formed by dispersion of the LLC lipid carrier disclosed herein in pharmaceutically or cosmetically acceptable carriers or diluents, including but not limited to water or any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavouring agents, dyes, and such similar materials and combinations thereof as would be known to persons skilled in the art (Remington's, 1990). Except insofar as any conventional pharmaceutically or cosmetically acceptable carrier is incompatible with the active ingredient, use in the therapeutic, pharmaceutical and cosmetic compositions is contemplated. The compositions used in the present disclosure may comprise different types of carriers depending on whether it is to be administered in solid, liquid or aerosol form, and whether it need to be sterile for such routes of administration as injection.

In embodiments, the amount of the polar medium (e.g., water) in the LLC lipid nanoparticle composition disclosed herein is at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% by weight. In some embodiments, the amount of the polar medium (e.g., water) in the LLC lipid nanoparticle composition disclosed herein is from about 80% to about 99% by weight, for example about 80%, about 82%, about 84%, about 86%, about 88%, about 90%, about 91%, about 92%, about 93%, bout 94%, about 95%, about 96%, about 97%, about 98%, or about 99% by weight. In some embodiments, the amount of the polar medium (e.g., water) in the LLC lipid nanoparticle composition disclosed herein is about 90% by weight.

The nanostructure of the LLC lipid carrier may be determined by any technique known to the person skilled in the relevant art. For example, the structure may be determined using small angle X-ray scattering (SAXS).

In embodiments, the mesophase transition of the LLC lipid carrier upon exposure to a drop in pH is a transition caused by a drop in pH below pH 10, below pH 9, below pH 8, or below pH 7. In embodiments, the LLC lipid carrier disclosed herein may undergo mesophase transition at a pH 4-pH 7, pH 5-7, or pH 5-6.5, for example pH 5.5-6.

In embodiments, the mesophase transition upon exposure to a drop in pH is a transition caused by a drop in pH of between 1 to 4 pH units.

In embodiments, the mesophase transition upon exposure to a drop in pH is a transition to a mesophase of lower interfacial curvature than the mesophase which the lipid carrier occupied prior to the pH drop.

In embodiments, the mesophase transition upon exposure to a drop in pH is a transition to a mesophase corresponding to lower CPP values.

In embodiments, the mesophase transition upon exposure to a drop in pH is a transition from one mesophase to another which follows the general mesophase transition order of L₂→H₂→Q₂→L₃. In embodiments, the phase transition is H₂→Q₂.

In embodiments, the nanostructure of the nonlamellar lyotropic liquid crystalline phase (the LLC lipid carrier) is hexagonal (H₂) or cubic (Q₂). In embodiments, the nonlamellar lyotropic liquid crystalline phase is hexagonal at a pH above about pH 10, about pH 9, about pH 8, or about pH 7. In embodiments, the nonlamellar lyotropic liquid crystalline phase is hexagonal at a pH of about pH 7 or higher, such as about pH 7.4 or higher, or about pH 7.5 or higher. In embodiments, the nonlamellar lyotropic liquid crystalline phase is cubic at a pH of below about pH 7.5, such as about pH 7.4 or lower.

In preferred embodiments, the nonlamellar lyotropic liquid crystalline phase transitions from hexagonal phase at a pH above about pH 8 to cubic phase at a pH of below about pH 7.5. In certain embodiments, the nonlamellar lyotropic liquid crystalline phase transitions from hexagonal phase at a pH above about pH 7.5 to cubic phase at a pH of below about pH 7. In certain embodiments, the nonlamellar lyotropic liquid crystalline phase transitions from hexagonal phase at a pH above about pH 6.5 to cubic phase at a pH of below about pH 6.5. In preferred embodiments, the nonlamellar lyotropic liquid crystalline phase undergoes a transition from predominantly hexagonal phase to predominantly cubic phase at a pH of below about pH 7. In some embodiments, the transition is complete within 0.5 pH values.

In preferred embodiments, the nonlamellar lyotropic liquid crystalline phase of the lipid carrier undergoes a transition from predominantly hexagonal phase at pH 7 to predominantly cubic phase at pH 5.5-6.0. Put another way, the lipid carrier undergoes a transition to a mesophase of lower interfacial curvature; and/or to a mesophase corresponding to lower CPP (critical packing parameter) values; and/or from one mesophase to another which is a transition in the general order of L₂→H₂→Q₂→L₃, although not necessarily a transition through each of these mesophases.

Advantageously, pH range 5.5-6.0 is a pathologically relevant pH range that has been reported to be found in tumours, infection sites of certain bacteria and fungi, and subcellular (intracellular) organelles, and is distinctly lower than healthy tissue (around pH 7). The present disclosure is predicated, at least in part, on the realisation that structural behaviour of the lipid carrier may be manipulated, using the amino lipid disclosed herein, to provide for a carrier particle which is capable of undergoing a phase change in response to a pH, such as a pH of between 5 and 7. For example, the nonlamellar lyotropic liquid crystalline phase may undergo a phase change to a mesophase of lower interfacial curvature in response to a reduction of pH from an ambient pH of around 7 to 7.5 to a decreased pH of below 7, such as a pH of between 5 and 7, for example between 5.5 and 6.0. As a reduced pH range may be found in tumours, infection sites of certain bacteria and fungi, and subcellular (intracellular) organelles, the LLC lipid carrier disclosed herein is believed to undergo the phase transition at such sites. For example, the LLC lipid nanoparticles disclosed herein may undergo a phase transition from the hexagonal phase at pH 7 to the cubic phase at pH 5.5-6.0.

### LLC Lipid Carrier Comprising an Active Agent

In embodiments, the LLC lipid carrier disclosed herein comprises an active agent. In embodiments, the active agent is encapsulated in the LLC lipid carrier.

In the context of the present disclosure, the encapsulating or including an active agent within the LLC lipid carrier means that when the encapsulated active agent is placed into the biological system most of the agent is not released immediately but rather the release is either slow and facilitated by diffusion of the agent through the lipid carrier, or the release is facilitated by a phase transition of the LLC lipid carrier.

In embodiments, the active agent is a pharmaceutically or cosmetically active agent. In embodiments, the active agent is selected from the group consisting of peptides, proteins, enzymes, small molecule drugs, and nucleic acids.

For example, suitable active agent include radionuclides, imaging agents, polymers, antibiotics, fungicides, metal-containing nanoparticles, anti-inflammatory agents, anti-cancer agents, cardiovascular agents, anti-anxiety agents, hormones, growth factors, steroidal agents, gene expression modifiers, knockdown agents, siRNA, RNAi agents, DNA, mRNA, dicer substrates, miRNA, shRNA, antisense oligonucleotides, aptamers, and microbially derived toxins.

In embodiments, the active agent is a small molecule drug. In preferred embodiments, the active agent is an anti-cancer chemotherapy drug. Non-limiting examples of anti-cancer chemotherapy drugs include temozolomide, dacarbazine, carmustine, lomustine, paclitaxel, docetaxel, vincristine, vinblastine, vinorelbine, etoposide, teniposide, topotecan, irinotecan, doxorubicin, daunomycin, epirubicin, idarubicin, methotrexate, cytarabine, gemcitabine, capecitabine, cisplatin, carboplatin, cyclophosphamide, oxaliplatin, or a mixture thereof, although without limitation thereto.

In some embodiments, the anti-cancer chemotherapy drug is a topoisomerase I inhibitor, such as, for example, camptothecin, irinotecan, and SN-38, preferably SN-38.

In embodiments, the active agent is a an antibiotic.

In embodiments, the active agent is a fungicide.

In embodiments, the active agent is an oligonucleotide or a nucleic acid.

In embodiments, the active agent is a cosmetically active agent.

As described above, the LLC lipid carrier disclosed herein may undergo mesophase transition in response to a predefined pH (e.g., a drop in pH), such as a pH of below 10, pH of below 9, pH of below 8, pH below 7.5, pH below 7; preferably at a pH 4-pH 7, pH 5-7, or pH 5-6.5, for example pH 5.5-6.

In embodiments, the LLC lipid carrier disclosed herein undergoes a transition to a mesophase of lower interfacial curvature in response to a drop in pH. For example, a drop in pH may lead to a phase transition of the LLC lipid carrier disclosed herein to phases with lower critical packing parameter (CPP), such as a transition from predominantly hexagonal phase (e.g., H₂), to predominantly cubic phase (e.g., Q₂). For example, the phase transition may be L₂→H₂→Q₂→L₃ (i.e., CPP high to low). In embodiments, the phase transition is H₂→Q₂.

In embodiments, the LLC lipid carrier disclosed herein undergoes a transition to a mesophase of lower interfacial curvature at around pH 10, around pH 9, around pH 8 around pH 7, or around pH 6. In embodiments, the LLC lipid carrier disclosed herein undergoes a transition to a mesophase of lower interfacial curvature at a pH below 10, pH below 9, pH below 8, or pH below 7, for example at pH 4.0-pH 7.0, pH 5-pH 7, or pH 5.5-pH 6.5. In embodiments, the LLC lipid carrier disclosed herein undergoes a transition to a predominantly cubic phase at a pH below 10, pH below 9, pH below 8, or pH below 7, for example at pH 4-pH 7, pH 5-pH 7, or pH 5.5-pH 6.5. In embodiments, the LLC lipid carrier disclosed herein undergoes a transition from a predominantly hexagonal phase to a predominantly cubic phase at a pH below 10, pH below 9, pH below 8, or pH below 7, for example at pH 4-pH 7, pH 5-pH 7, or pH 5.5-pH 6.5. The transition may be from predominantly hexagonal phase at around pH 7 or higher (e.g., pH 7.4 or higher) to a predominantly cubic phase at a pH below 7, for example at pH 4.0-pH 7.0, pH 5-pH 7, or pH 5.5-pH 6.5. In some embodiments, the LLC lipid carrier disclosed herein undergoes a mesophase transition from a hexagonal phase at about pH 7 or higher (e.g., pH 7.4 or higher) to a cubic phase at about pH 4.0-pH 7.0, pH 5-pH 7, or pH 5.5-pH 6.5. In certain embodiments, the mesophase transition is from an inverse hexagonal phase at about pH 7 or higher (e.g., pH 7.4 or higher) to a bicontinuous cubic phase at about pH 4.0-pH 7.0, pH 5-pH 7, or pH 5.5-pH 6.5.

Previous studies have shown that a hexagonal structure host will release an encapsulated guest much slower than a corresponding cubic structure host. In agreement with those previous studies but without wishing to be bound by theory, the LLC lipid carrier disclosed herein, is believed to release an encapsulated active agent when transitioning from hexagonal to cubic phase structure at low pH. Accordingly, the encapsulated agent will have a slow release profile while the lipid carrier is in an environment with an ambient pH, e.g. physiological of about 7-7.5, and a faster release profile once the lipid carrier is in an environment with a reduced pH, e.g. a pH of below 7 as found, for example a pH of between 7 and 5. Such a reduced pH range may be found in environments including tumour sites, infection sites of certain bacteria and fungi, and subcellular organelles.

### Compositions of the LLC Lipid Carrier Comprising an Active Agent

In a third aspect, there is provided a cosmetic composition comprising the LLC lipid carrier of the first aspect and a cosmetically active agent, and a cosmetically acceptable carrier, diluent and/or excipient.

In a fourth aspect, there is provided a pharmaceutical composition comprising the LLC lipid carrier of the first aspect and a pharmaceutically active agent, and a pharmaceutically acceptable carrier, diluent and/or excipient.

Suitably, the cosmetically or pharmaceutically acceptable carrier, diluent and/or excipient may be or include one or more of diluents, solvents, pH buffers, binders, fillers, emulsifiers, disintegrants, polymers, lubricants, oils, fats, waxes, coatings, viscosity-modifying agents, glidants and the like.

Diluents may include one or more of microcrystalline cellulose, lactose, mannitol, calcium phosphate, calcium sulfate, kaolin, dry starch, powdered sugar, and the like. Binders may include one or more of povidone, starch, stearic acid, gums, hydroxypropylmethyl cellulose and the like. Disintegrants may include one or more of starch, croscarmellose sodium, crospovidone, sodium starch glycolate and the like. Solvents may include one or more of ethanol, methanol, isopropanol, chloroform, acetone, methylethyl ketone, methylene chloride, water and the like. Lubricants may include one or more of magnesium stearate, zinc stearate, calcium stearate, stearic acid, sodium stearyl fumarate, hydrogenated vegetable oil, glyceryl behenate and the like. A glidant may be one or more of colloidal silicon dioxide, talc or cornstarch and the like. Buffers may include phosphate buffers, borate buffers and carbonate buffers, although without limitation thereto. Fillers may include one or more gels inclusive of gelatin, starch and synthetic polymer gels, although without limitation thereto. Coatings may comprise one or more of film formers, solvents, plasticizers and the like. Suitable film formers may be one or more of hydroxypropyl methyl cellulose, methyl hydroxyethyl cellulose, ethyl cellulose, hydroxypropyl cellulose, povidone, sodium carboxymethyl cellulose, polyethylene glycol, acrylates and the like. Suitable solvents may be one or more of water, ethanol, methanol, isopropanol, chloroform, acetone, methylethyl ketone, methylene chloride and the like. Plasticizers may be one or more of propylene glycol, castor oil, glycerin, polyethylene glycol, polysorbates, and the like.

With regard to a pharmaceutically acceptable carriers, diluents and/or excipients, reference is made to the Handbook of Excipients 6th Edition, Eds. Rowe, Sheskey & Quinn (Pharmaceutical Press), which provides non-limiting examples of excipients which may be useful according to the disclosure.

Cosmetic compositions disclosed herein may further comprise nutrients, such as carnitine, iron as ferrous, ferric salts or elemental iron, iodine, folic acid, vitamins or micronutrients.

Cosmetic compositions may also comprise a colouring or flavouring agent.

It will be appreciated that the choice of cosmetically or pharmaceutically acceptable carriers, diluents and/or excipients will, at least in part, be dependent upon the mode of administration of the formulation. By way of example only, the composition may be in the form of a tablet, capsule, caplet, powder, an injectable liquid, a suppository, a slow release formulation, an osmotic pump formulation or any other form that is effective and safe for administration.

In embodiments, the cosmetic composition or the pharmaceutical composition is a liquid dispersion of the LLC lipid carrier comprising a cosmetically or pharmaceutically active agent, respectively. The liquid dispersion may be an aqueous dispersion. The liquid dispersion may be encapsulated within standard capsules known for delivery of liquid formulations. The liquid dispersion may be formulated for delivery via injection, or for topical administration, or for subcutaneous administration.

### Methods of Delivery of an Active Agent Using the LLC Lipid Carrier

In a fifth aspect, there is provided a method of delivering an active agent to a biological system including the step of administering to the biological system the LLC lipid carrier of the first aspect, the LLC lipid nanoparticle composition of the second aspect, or the composition of the third or fourth aspect.

In a sixth aspect, there is provided a method of controlled release of an active agent comprising the steps of forming the LLC lipid carrier of the first aspect, the LLC lipid nanoparticle composition of the second aspect, or the composition of the third or fourth aspect, and administering the lipid carrier or the composition to a biological system comprising a target area having a predefined pH to thereby achieve a preferential release of the active agent at the target area.

As discussed, the LLC lipid carried disclosed herein may provide for a desirable release profile for a host active agent due to the trapping of the active agent within the complex internal architecture of the nonlamellar lyotropic liquid crystalline phase at ambient pH, such as a physiological pH of between about 7.4 (e.g. between 7.35 to 7.45).

The target area may be any area to which it is desirable to deliver the active agent. The target area may be within a biological sample or a tissue or fluid of a mammal subject. For example, the target area may be a tumour, a tissue infected with a bacterial infection or a tissue infected with a fungal infection. The target area may be a population of cells in a subject or in a cell culture. For example, the target area may be a subcellular organelle.

### Therapeutic and Diagnostic Applications of the LLC Lipid Carrier Comprising an Active Agent

In a seventh aspect, there is provided a method of treatment or prevention of a disease, disorder or condition in a mammal, comprising administering to the mammal a therapeutically effective amount of the LLC lipid carrier of the first aspect, the LLC lipid nanoparticle composition of the second aspect, or the composition of the third or fourth aspect.

In an eighth aspect, there is provided the LLC lipid carrier of the first aspect, the LLC lipid nanoparticle composition of the second aspect, or the composition of the third or fourth aspect, for use in the treatment or prevention of a disease, disorder or condition.

In a ninth aspect, there is provided a use of the LLC lipid carrier of the first aspect, the LLC lipid nanoparticle composition of the second aspect, or the composition of the third or fourth aspect, in the manufacture of a medicament for the treatment of a disease, disorder or condition.

In a tenth aspect, there is provided a method of diagnosing a disease, disorder or condition in a mammal including the step of administering the LLC lipid carrier of the first aspect, the LLC lipid nanoparticle composition of the second aspect, or the composition of the third or fourth aspect, wherein the active agent is a labelled active agent, to the mammal or to a biological sample obtained from the mammal to facilitate diagnosis of the disease disorder or condition in the mammal

As generally used herein, the terms “administering” or “administration”, and the like, describe the introduction of the relevant particle or composition to a mammal such as by a particular route or vehicle. Routes of administration may include topical, parenteral and enteral which include oral, buccal, sub-lingual, nasal, anal, gastrointestinal, subcutaneous, intramuscular and intradermal routes of administration, although without limitation thereto. The compounds, compositions, and methods of the invention can be used both for delivery to a particular site of administration or for systemic delivery.

By “treat”, “treatment” or treating” is meant administration of the relevant particle or composition to a subject to at least ameliorate, reduce or suppress existing signs or symptoms of the disease, disorder or condition experienced by the subject. to the extent that the medical condition is improved according to clinically acceptable standard(s). For example, “to treat a bacterial or fungal infection” means to reduce the infection, or eradicate the infection, or relieve symptoms of the infection in a patient, wherein the improvement and relief are evaluated with a clinically acceptable standardized test and/or an empirical test, including swab sample testing and the like.

By “prevent”, “preventing” or “preventative” is meant prophylactically administering the relevant particle or composition to a subject who does not exhibit signs or symptoms of a disease disorder or condition, but who is expected or anticipated to likely exhibit such signs or symptoms in the absence of prevention. Preventative treatment may at least lessen or partly ameliorate expected symptoms or signs.

As used herein, “effective amount” or “therapeutically effective amount” refers to the administration of an amount of the relevant particle or composition sufficient to prevent the occurrence of symptoms of the condition being treated, or to bring about a halt in the worsening of symptoms or to treat and alleviate or at least reduce the severity of the symptoms. The effective amount will vary in a manner which would be understood by a person of skill in the art with patient age, sex, weight etc. An appropriate dosage or dosage regime can be ascertained through routine trial or based on current treatment regimens for the active being delivered via the particle of the first or second aspect.

As used herein, the terms “subject” or “individual” or “patient” may refer to any subject, particularly a vertebrate subject, and even more particularly a mammalian subject, for whom therapy is desired. Suitable vertebrate animals include, but are not restricted to, primates, avians, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes). A preferred subject is a human in need of treatment for a disease, disorder or condition as described herein. However, it will be understood that the aforementioned terms do not imply that symptoms are necessarily present. In one embodiment, the subject is a human being treated for a bacterial or fungal infection, particularly a gram-negative bacterial infection.

As used herein, the terms “co-therapy” and “combination therapy” shall mean treatment of a subject in need thereof by administering one or more particles or compositions as described herein and one or more agents for treating a disease, disorder or condition by any suitable means, simultaneously, sequentially, separately or in a single pharmaceutical formulation or combination. When administered in separate dosage forms, the number of dosages administered per day for each compound may be the same or different. The relevant particle or composition and one or more active agents for treating a disease, disorder or condition may be administered via the same or different routes of administration.

As discussed above, the LLC lipid carrier of a first aspect can comprise an active agent, which can be pharmaceutically active. The lipid carrier may comprise two or more different pharmaceutically active agent, for example if the agents are effective against the same or different targets. The agents may have synergistic activity. For example, delivery of different agents may be advantageous when treating a multiple-strain bacterial infection, or when targeting tumour cells that express two or more different molecular targets.

The LLC lipid carrier (e.g., LLC lipid nanoparticles) disclosed herein are particularly suited for controlled delivery of an active agent, which can be pharmaceutically or cosmetically active. Accordingly, the present disclosure also provides methods of treatment, prevention or diagnosis of a disease, disorder or condition that include administering to a subject the LLC lipid carrier (e.g., LLC lipid nanoparticles) disclosed herein, the LLC lipid nanoparticle compositions disclosed herein, or the cosmetic or pharmaceutical composition disclosed herein.

As discussed above, the LLC lipid carrier disclosed herein may undergo a phase change in response to a pH, for example a reduction of pH from physiological pH of around 7.4 (e.g. 7.35 to 7.45) to a pH of between 7 and 5. Such reduced pH range may be found in environments including a tumour, infection site of certain bacteria and fungi, and subcellular organelles, and can trigger a phase change of the disclosed nonlamellar lyotropic liquid crystalline phase. The phase change, in turn, may facilitate the release of the encapsulated active agent.

Accordingly, in some embodiments, the disease, disorder or condition suitable for the treatment, prevention or diagnosis by the methods disclosed herein is a cancer or a bacterial or fungal infection.

In some embodiments, the disease, disorder or condition suitable for the treatment, prevention or diagnosis by the methods disclosed herein is a tumour or other malignancy. As used herein, cancers tumours and malignancies, refer to diseases disorders or conditions, or to cells or tissues associated with the diseases, disorders or conditions, characterized by aberrant or abnormal cell proliferation, differentiation and/or migration often accompanied by an aberrant or abnormal molecular phenotype that includes one or more genetic mutations or other genetic changes associated with oncogenesis, expression of tumour markers, loss of tumour suppressor expression or activity and/or aberrant or abnormal cell surface marker expression. In general embodiments, cancers, tumours and malignancies may include sarcomas, lymphomas, leukemias, solid tumours, blastomas, gliomas, carcinomas, melanomas and metastatic cancers, although without limitation thereto. A more comprehensive listing of cancers tumours and malignancies may be found at the National Cancer Institutes website http://www.cancer.gov/cancertopics/types/alphalist.

In some embodiments, the disease, disorder or condition is one which is a bacterial infection. In one embodiment, the disease, disorder or condition is caused by, or is associated with, a pathogen. The pathogen may be a bacterium or a fungus capable of infecting a mammal.

In embodiments wherein the infection suitable for being treated, prevented or diagnosed by the methods disclosed herein is a gram-negative infection then the bacteria may be selected from the group consisting of Enterobacteriaceae, Pseudomonas, Vibrio, Campylobacter, Legionella, Neisseria, Hemophilus and Bartonella.

In embodiments, the gram-negative bacteria may be selected from the group consisting of E. coli, Pseudomonas aeruginosa, Klebsiella, Acinetobacter baumannii, Neisseria gonorrhoeae, and Enterobacteriaceae.

In embodiments wherein the infection suitable for being treated, prevented or diagnosed by the methods disclosed herein is a gram-positive infection then the bacteria may be selected from the group consisting of Staphylococci, Streptococci, Pneumococci, Enterococci, Bacilli, Clostridia, Corynebacterium, Listeria and Actinomyces.

Non-limiting examples of pathogenic bacteria include Staphylococcus aureus, Helicobacter pylori, Bacillus anthracis, Bordatella pertussis, Corynebacterium diptheriae, Clostridium tetani, Clostridium botulinum, Streptococcus pneumoniae, Streptococcus pyogenes, Listeria monocytogenes, Hemophilus influenzae, Pasteurella multicida, Shigella dysenteriae, Mycobacterium tuberculosis, Mycobacterium leprae, Mycoplasma pneumoniae, Mycoplasma hominis, Neisseria meningitidis, Neisseria gonorrhoeae, Rickettsia rickettsii, Legionella pneumophila, Klebsiella pneumoniae, Pseudomonas aeruginosa, Propionibacterium acnes, Treponema pallidum, Chlamydia trachomatis, Vibrio cholerae, Salmonella typhimurium, Salmonella typhi, Borrelia burgdorferi and Yersinia pestis, although without limitation thereto.

Non-limiting examples of fungi include Candida and Aspergillus species, although without limitation thereto. In embodiments, the fungus associated with or causing the disease, disorder or condition is selected from the group consisting of C. albicans, C. glabrata, C. parapsilosis, C. tropicalis, C. dublinensis, C. krusei, C. lusitaniae, C. Auris, and A. fumigatus.

As discussed above, phase change of the LLC lipid carrier disclosed herein and associated release of the encapsulated active agent may occur in subcellular (intracellular) organelles. Typically, delivery of therapeutic nucleic acids hinges on the delivery of the nucleic acids into cells where they exert their mode of action. Accordingly, any condition suitable for therapy by oligonucleotides or nucleic acids may be treated by the LLC lipid carrier disclosed herein that comprises an active agent. Suitable oligonucleotides or nucleic acids may be selected from gene expression modifiers, knockdown agents, small interfering RNA (siRNA), RNA interference (RNAi), mRNA, DNA, antisense oligonucleotides, functional nucleic acids such as ribozymes, aptamers, and spiegelmers, dicer substrates, micro RNA (miRNA), and short hairpin RNA (shRNA).

Where the active agent is a labelled active agent, the lipid carrier of the second aspect comprising the active agent, or the composition of the third or fourth aspect comprising the active agent may be used for detection or diagnosis of the disease, disorder or condition discussed above.

Typically, the detection or diagnosis will be performed in a mammal or in a cell population.

In embodiments, the labelled active agent is a contrast agent.

Disclosed herein are the following numbered embodiments.

Embodiment 1. A lipid compound of Formula (I):

Cyc-L-R (I),
wherein Cyc is a nitrogen heterocycle or heteroaryl;
L is an amido-linker; and
R is a C10 to C44 carbon chain.

Embodiment 2. The lipid compound of embodiment 1, wherein Cyc is selected from a 5- or 6-membered nitrogen-heterocyclyl or -heteroaryl group.

Embodiment 3. The lipid compound of embodiment 1 or embodiment 2, wherein Cyc is selected from the group consisting of piperidinyl, piperazinyl, pyrrolinyl, pyrrolidinyl, pyrazolidinyl, morpholinyl, imidazolinyl, imidazolidinyl, pyrrolinyl, pyrazolinyl, thiazolidinyl, thiomorpholinyl, tetrahydropyridyl, dihydropyridyl, tetrahydropyrinidinyl, pyrimidinyl, pyridyl, pyrrolyl, imidazolyl, pyrazolyl, oxazolyl, oxadiazolyl, pyrazinyl, tetrazolyl, thiazolyl, isoxazolyl, isothiazolyl, isoxazolonyl, triazolyl, oxadiazolyl, thiadiazolyl, and pyridazinyl.

Embodiment 4. The lipid compound of any one of the preceding embodiments, wherein L comprises an optional carbon chain between Cyc and the amide group.

Embodiment 5. The lipid compound of embodiment 4, wherein the carbon chain between Cyc and the amide group, when present, is a C₁ to C₆ chain.

Embodiment 6. The lipid compound of any one of the preceding embodiments, wherein R is a C10 to C36 alkyl, alkenyl or alkynyl chain.

Embodiment 7. The lipid compound of any one of the preceding embodiments, wherein R is a C12 to C24 alkyl or alkenyl chain.

Embodiment 8. The lipid compound of any one of the preceding embodiments, wherein R is a C12 to C18 alkenyl chain.

Embodiment 9. The lipid compound of any one of the preceding embodiments, wherein R is optionally interrupted by one or more heteroatoms.

Embodiment 10. The lipid compound of any one of the preceding embodiments, wherein the amino lipid compound of Formula (I) is a compound of Formula (Ib):

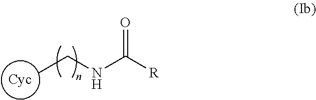

wherein Cyc is a nitrogen heterocycle or heteroaryl;
R is a C12 to C24 alkyl or alkenyl chain; and
n is an integer from 1 and 6.

Embodiment 11. The lipid compound of embodiment 10, wherein Cyc is a 6-membered nitrogen-heterocycle or -heteroaryl.

Embodiment 12. The lipid compound of embodiment 11, wherein Cyc is selected from the group consisting of pyridyl, pyrimidinyl, piperidinyl, piperazinyl, morpholinyl, thiomorpholinyl, tetrahydropyridyl, dihydropyridyl, tetrahydropyrinidinyl, pyrazinyl and pyridazinyl.

Embodiment 13. The lipid compound of embodiment 12, wherein Cyc is selected from the group consisting of pyridyl, pyrimidinyl, piperidinyl, piperazinyl, morpholinyl, and pyrazinyl.

Embodiment 14. The lipid compound of any one of embodiment 10 to embodiment 13, wherein n is an integer from 1 to 4.

Embodiment 15. The lipid compound of any one of embodiment 10 to embodiment 14, wherein R is C12 to C24 alkenyl.

Embodiment 16. The lipid compound of embodiment 14, wherein R is C14 to C22 alkenyl.

Embodiment 17. The lipid compound of any one of the preceding embodiments, wherein R is selected from the group consisting of oleyl, linoleoyl, linolenoyl, phytanoyl, and farnesoyl.

Embodiment 18. The lipid compound of any one of embodiment 10 to embodiment 17, wherein R is optionally interrupted by one or more heteroatoms.

Embodiment 19. The lipid compound of any one of the preceding embodiments, wherein the lipid compound is selected from the group consisting of

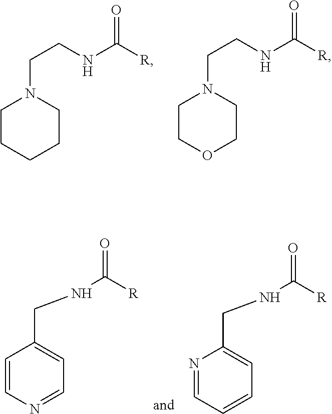

wherein, R is as defined in any one of the preceding embodiments.

Embodiment 20. The lipid compound of embodiment 19, wherein the lipid compound is selected from the group consisting of

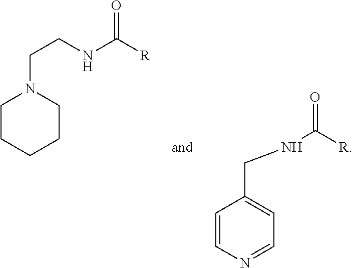

Embodiment 21. The lipid compound of any one of the preceding embodiments, wherein R is:

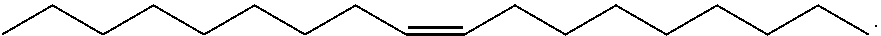 [SMILES: CCCCCCCC/C=C\CCCCCCCC]

Embodiment 22. A lipid carrier comprising a lipid having an amido-linker, the lipid carrier adapted to undergo a mesophase transition upon exposure to a drop in pH.

Embodiment 23. The lipid carrier of embodiment 22, further comprising a structural lipid compound, wherein the structural lipid compound is amphiphilic.

Embodiment 24. The lipid carrier of embodiment 23, wherein the structural lipid compound comprises a hydrophobic tail group selected from the group consisting of oleyl, linoleoyl, linolenoyl, phytanoyl, famesoyl or extended aliphatic hydrophobic.

Embodiment 25. The lipid carrier of embodiment 24, wherein the hydrophobic tail group is selected from oleyl, linoleoyl, and phytanoyl.

Embodiment 26. The lipid carrier of embodiment 24 or embodiment 25, wherein the hydrophobic tail group is oleyl.

Embodiment 27. The lipid carrier of any one of embodiment 23 to embodiment 26, wherein the structural lipid compound is monoolein (glycerol monooleate).

Embodiment 28. The lipid carrier of any one of embodiment 22 to embodiment 27, wherein the lipid carrier is a nonlamellar lyotropic liquid crystalline phase lipid carrier.

Embodiment 29. The lipid carrier of embodiment 28, wherein the nonlamellar lyotropic liquid crystalline phase lipid carrier is a self-assembled nonlamellar lyotropic liquid crystalline phase lipid carrier.

Embodiment 30. The lipid carrier of embodiment 28 or embodiment 29, wherein the nonlamellar lyotropic liquid crystalline phase lipid carrier is in the form of a nanoparticle.

Embodiment 31. The lipid carrier of any one of embodiment 28 to embodiment 30, wherein the nonlamellar lyotropic liquid crystalline phase lipid carrier further comprises a stabilizer.

Embodiment 32. The lipid carrier of embodiment 31, wherein the stabilizer is a stabilizing polymer.

Embodiment 33. The lipid carrier of embodiment 32, wherein the stabilising polymer is a non-ionic triblock copolymer.

Embodiment 34. The lipid carrier of embodiment 32 or embodiment 33, wherein the stabilising polymer is Pluronic F127 (Poloxamer 407) or Poloxamer 80.

Embodiment 35. The lipid carrier of any one of embodiment 22 to embodiment 34, wherein the lipid having an amido-linker is present at a suitable wt % of the total lipid content of the lipid carrier to provide for the mesophase transition at a selected pH.

Embodiment 36. The lipid carrier of any one of embodiment 22 to embodiment 35, wherein the lipid having an amido-linker comprises about 5% to about 50 wt % of the total lipid content of the lipid carrier.

Embodiment 37. The lipid carrier of any one of embodiment 22 to embodiment 36, wherein the lipid having an amido-linker is the lipid compound of any one of embodiment 1 to embodiment 21.

Embodiment 38. The lipid carrier of any one of embodiment 28 to embodiment 37, wherein the nonlamellar lyotropic liquid crystalline phase is a hexagonal or a cubic nonlamellar lyotropic liquid crystalline phase.

Embodiment 39. The lipid carrier of embodiment 38, wherein the nonlamellar lyotropic liquid crystalline phase is a hexagonal nonlamellar lyotropic liquid crystalline phase at a pH above about pH 8.

Embodiment 40. The lipid carrier of embodiment 38 or embodiment 39, wherein the nonlamellar lyotropic liquid crystalline phase is a cubic nonlamellar lyotropic liquid crystalline phase at a pH below about pH 7.5.

Embodiment 41. The lipid carrier of embodiment 38 to embodiment 40, wherein the nonlamellar lyotropic liquid crystalline phase is a hexagonal nonlamellar lyotropic liquid crystalline phase at a pH above about pH 8, and a cubic nonlamellar lyotropic liquid crystalline phase at a pH below about pH 7.5.

Embodiment 42. The lipid carrier of embodiment 38 or embodiment 39, wherein the nonlamellar lyotropic liquid crystalline phase is a hexagonal nonlamellar lyotropic liquid crystalline phase at a pH above about pH 7.5, and a cubic nonlamellar lyotropic liquid crystalline phase at a pH below about pH 7.

Embodiment 43. The lipid carrier of embodiment 38 or embodiment 39, wherein the nonlamellar lyotropic liquid crystalline phase is a hexagonal nonlamellar lyotropic liquid crystalline phase at a pH above about pH 7, and a cubic nonlamellar lyotropic liquid crystalline phase at a pH below about pH 6.5.

Embodiment 44. The lipid carrier of embodiment 35 to embodiment 37, wherein the nonlamellar lyotropic liquid crystalline phase is a hexagonal nonlamellar lyotropic liquid crystalline phase at a pH above about pH 6.5, and a cubic nonlamellar lyotropic liquid crystalline phase at a pH of about pH 6.5 or below.

Embodiment 45. The lipid carrier of any one of embodiment 22 to embodiment 44, further comprising an active agent.

Embodiment 46. The lipid carrier of embodiment 45, wherein the active agent is pharmaceutically or cosmetically active.

Embodiment 47. The lipid carrier of embodiment 45 or embodiment 46, wherein the active agent is selected from the group consisting of peptides, proteins, enzymes, small molecule drugs, and nucleic acids.

Embodiment 48. The lipid carrier of any one of embodiment 45 to embodiment 47, wherein the active agent is selected from the group consisting of radionuclides, imaging agents, polymers, antibiotics, fungicides, metal-containing nanoparticles, anti-inflammatory agents, anti-tumor agents, cardiovascular agents, anti-anxiety agents, hormones, growth factors, steroidal agents, gene expression modifiers, knockdown agents, siRNA, RNAi agents, mRNA, DNA, dicer substrates, miRNA, shRNA, antisense oligonucleotides, aptamers, and microbially derived toxins.

Embodiment 49. The lipid carrier of any one of embodiment 45 to embodiment 48, wherein the active agent is a topoisomerase I inhibitor.

Embodiment 50. The lipid carrier of embodiment 49, wherein the a topoisomerase I inhibitor is selected from camptothecin, irinotecan, and SN-38.

Embodiment 51. A cosmetic composition comprising the lipid carrier of any one of embodiment 22 to embodiment 46, and a cosmetically acceptable carrier, diluent and/or excipient.

Embodiment 52. A pharmaceutical composition comprising the lipid carrier of any one of embodiment 22 to embodiment 50, and a pharmaceutically acceptable carrier, diluent and/or excipient.

Embodiment 53. The cosmetic composition of embodiment 51, or the pharmaceutical composition of embodiment 52, wherein the composition is a composition for injection.

Embodiment 54. The cosmetic composition of embodiment 51, or the pharmaceutical composition of embodiment 52, wherein the composition is a composition for topical administration.

Embodiment 55. The cosmetic composition of embodiment 51, or the pharmaceutical composition of embodiment 52, wherein the composition is a composition for subcutaneous administration.

Embodiment 56. A method of delivering an active agent to a biological system including the step of administering to the biological system the lipid carrier of any one of embodiment 45 to embodiment 50, or the composition of any one of embodiment 51 to embodiment 55.

Embodiment 57. The method of embodiment 56, wherein the delivery of the active agent in the biological system is modified by a response of the lipid carrier to a predefined pH range.

Embodiment 58. The method of embodiment 57, wherein the response comprises a phase structure transition of the lipid carrier.

Embodiment 59. The method of embodiment 58, wherein the phase structure transition is a transition from hexagonal to cubic phase structure of the lipid carrier.

Embodiment 60. The method of embodiment 59, wherein the transition occurs at a pH of below about pH 8.

Embodiment 61. The method of embodiment 59 or embodiment 60, wherein the transition occurs at a pH of about pH 7.5 or below.

Embodiment 62. The method of any one of embodiment 59 to embodiment 61, wherein the transition occurs at a pH of about pH 7 or below.

Embodiment 63. The method of any one of embodiment 59 to embodiment 62, wherein the transition occurs at a pH of about pH 6.5 or below.

Embodiment 64. The method according to any one of embodiment 56 to embodiment 63, wherein the response provides for a preferential release of the active agent at the predefined pH range in the biological system.

Embodiment 65. A method of controlled release of an active agent comprising the steps of forming the lipid carrier of any one of embodiment 45 to embodiment 50, or the composition of any one of embodiment 51 to 55, and administering the lipid carrier or the composition to a biological system comprising a target area having a predefined pH to thereby achieve a preferential release of the active agent at the target area.

Embodiment 66. The method of embodiment 65, wherein the lipid carrier undergoes a phase structure transition in response to the predefined pH.

Embodiment 67. The method of embodiment 66, wherein the phase structure transition is a transition from hexagonal to cubic phase structure of the lipid carrier.

Embodiment 68. A method of treatment or prevention of a disease, disorder or condition in a mammal, comprising administering to the mammal a therapeutically effective amount of the lipid carrier of any one of embodiment 45 to embodiment 50, or the composition of any one of embodiment 51 to embodiment 55.

Embodiment 69. The lipid carrier of any one of embodiment 45 to embodiment 50, or the composition of any one of embodiment 51 to embodiment 55, for use in the treatment or prevention of a disease, disorder or condition.

Embodiment 70. Use of the lipid carrier of any one of embodiment 45 to embodiment 50, or the composition of any one of embodiment 51 to embodiment 55, in the manufacture of a medicament for the treatment of a disease, disorder or condition.

Embodiment 71. A method of diagnosing a disease, disorder or condition in a mammal including the step of administering the lipid carrier of any one of embodiment 45 to embodiment 50, or the composition of any one of embodiment 51 to embodiment 55, wherein the active agent is a labelled active agent, to the mammal or to a biological sample obtained from the mammal to facilitate diagnosis of the disease disorder or condition in the mammal.

Embodiment 72. The method as described in embodiment 68 or embodiment 71, the lipid carrier or the pharmaceutical composition for use as described in embodiment 69, or the use as described in embodiment 70, wherein the disease, disorder or condition is a cancer or a bacterial or fungal infection.

Embodiment 73. The lipid carrier of any preceding embodiment wherein the water content of the lipid carrier itself is between about 20 to about 60 wt %.

The following experimental section describes in more detail the characterisation of certain of the compounds of the invention and their efficacy. The intention is to illustrate certain specific embodiments of the compounds of the invention and their efficacy without limiting the invention in any way.

### EXAMPLES

The present disclosure will now be further described with reference to the following non-limiting examples and with reference to the accompanying Figures.

### Materials

4-(Aminomethyl) pyridine, Picolylamine, 4-(2-Aminoethyl) morpholine, 1-(2-Aminoethyl) piperidine, N-(3-Dimethylaminopropyl)-N′-ethyl carbodiimide hydrochloride (EDC), Hydroxybenzotriazole (HOBt), Dimethyl aminopyridine (DMAP), Dichloromethane (DCM), n-hexane, ethyl acetate, sodium sulphate, sodium chloride, deuterated chloroform, deuterated dimethyl sulfoxide, deuterium oxide, and Pluronic F-127 were purchased from Sigma-Aldrich. Milli-Q water was used (18.2 18.2 MΩ·cm) for all aqueous preparations. Monoolein (MO) and oleic acid (OA) were obtained from Nu-chek-Prep, Inc (Elysian, MN, USA) with purity >99%. SN-38 was procured from Adooq bioscience.

### Example 1: Synthesis, Purification and Analysis of the Amino Lipids

The amino lipids disclosed herein comprise a hydrophilic head group comprising a nitrogen heterocycle or heteroaryl, conjugated to a hydrophobic carbon chain tail via an amido-linker. In the following examples, C18-oleyl tail was used and kept constant for all synthetic lipids but the head group and the length of the amido-linker were varied.

The amino lipids were synthesized utilizing an amide coupling reaction between oleic acid (OA) and amines carrying head groups. To a cooled OA solution (3.54 mmol, 1.0 eq) in DCM, EDC (3.9 mmol, 1.1 eq) was added. The solution was stirred for 30 min at 0-5° C. and to this cooled reaction mass amine (3.54 mmol, 1.0 eq) and DMAP (0.7 mmol, 0.2 eq) were added. The reaction temperature was slowly raised to room temperature (RT) and stirred at RT for 24 hours. The solvent was removed using a rotary evaporator, and a flash silica column used to purify the obtained crude material. The synthesised amino lipids were subjected to analysis by nuclear magnetic resonance (NMR) imaging (¹H) analysis. ¹H NMR spectra were obtained using a Bruker 300 Ultrashield™ NMR spectrometer.

### Results:

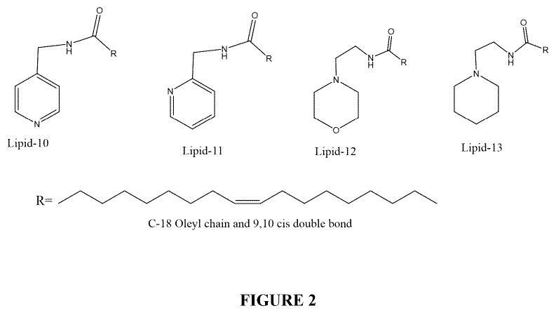

The physical state of the synthesised amino lipids was semi-solid at room temperature. In general, the reactions' yield was in a range of 60-75% and purity of 85-95%. Structure and purity of the synthesised amino lipids were confirmed by NMR (Data not shown). Exemplary structures synthesised by the method are shown in FIG. 2.

The following amino lipids were used or referred to in the examples below:

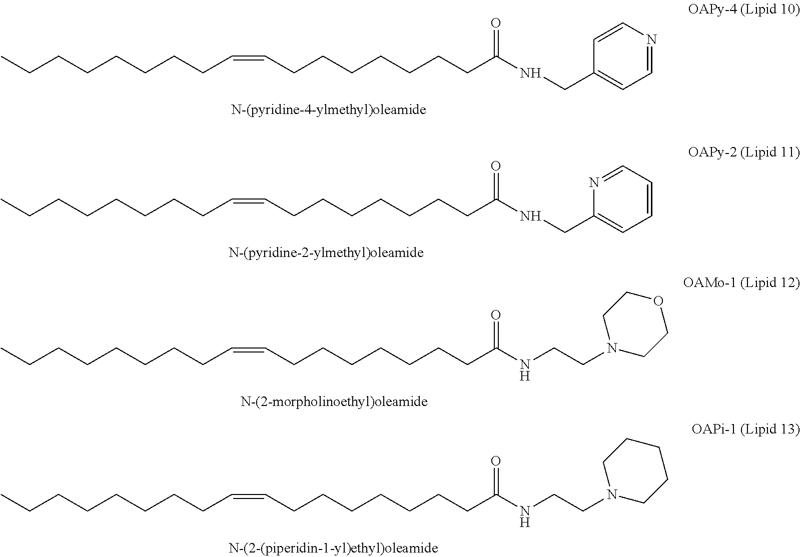 [SMILES: CCCCCCCC/C=C\CCCCCCCC(=O)NCc1ccncc1 ; CCCCCCCC/C=C\CCCCCCCC(=O)NCc1ccccn1 ; CCCCCCCC/C=C\CCCCCCCC(=O)NCCN1CCOCC1 ; CCCCCCCC/C=C\CCCCCCCC(=O)NCCN1CCCCC1]

### Example 2: Preparation of LLC Nanoparticles Comprising the Amino Lipid

The LLC lipid carrier disclosed herein may be in bulk phase or may be dispersed in the form of nanoparticles comprising the amino lipids disclosed herein and a structural amphiphilic lipid. The structural amphiphilic lipid may make up the bulk of the nanoparticles depending on the amount of the amino lipids used. In the following examples, monoolein (MO) was used as the structural amphiphilic lipid.

LLC nanoparticles consisting of an amino lipid and MO were formulated using a high throughput method as reported in Tran et al. 2016, which is incorporated herein by reference. The amount of amino lipid added to the formulation is represented by R_{MO}, which is defined as the wt/wt ratio of amino lipid to the total amount of lipid in the system, and may be referred to as RAL herein. Briefly, MO (20 mg/1 mL of ethanol) and amino lipid (20 mg/1 mL of ethanol) were mixed at an increasing ratio of amino lipid at 95:5, 90:10, 85:15, 80:20, 75:25, 70:30, 60:40, 50:50 to obtain R_{MO} values of 0.05, 0.1, 0.15, 0.2, 0.25, 0.3, 0.4 and 0.5. 1 mL of lipid solution was added to each well of a 96-well deep well block (Greiner Bio-One, Interpath Inc., Australia). The organic solvent was then removed using a centrifugal evaporator (GeneVac, NSW, Australia) to obtain a dried lipid mixture.

In these examples, Pluronic F-127 was used to stabilize the nanoparticles. Accordingly, to the dried lipid mixture, 1 mL of F127 (solution of 20 mg in 1 mL of DI water) was added. The resulting mixture was sonicated by a high throughput multiprobe sonicator (Q Sonica) to obtain an opaque dispersion.

The nanoparticles may also be prepared using a mixture of two or more amino lipids to fine tune their pH responsiveness.

### Results:

Various amounts of amino lipids from 5 wt % to 50 wt % were added to MO and dispersed them with Pluronic F-127. The amount of Pluronic F-127 was kept at 10 wt % of the total amount of lipids in the system. These formulations were all dispersed well with no visual sedimentation or phase separation.

### Example 3: Particle Size and Polydispersity Index of LLC Lipid Nanoparticles

The average hydrodynamic diameter and polydispersity index (PDI) of lipid nanoparticles were measured by dynamic light scattering (DLS) using a Malvern Zetasizer Nano ZS (Malvern Instruments, UK). All measurements were performed at 25° C. (n=3) by loading the lipid formulations (10 μL) into clear 96-well half-area polystyrene plates and diluting with 190 μL milli-Q water (Zhai et al. 2015).

### Results

Nanoparticle formulations described above were investigated for an average particle size and polydispersity index (PDI). The resulting values are represented in Tables 1 and 2, respectively.

TABLE 1
Particle Size in nm

| | Lipid-10 | Lipid-11 | Lipid-12 | Lipid-13 |
|---|---|---|---|---|
| R_{MO} | (OAPy-4) | (OAPy-2) | (OAMo-1) | (OAPi-1) |
| 0.05 | 217 ± 6 | 284 ± 2 | 214 ± 4 | 180 ± 3 |
| 0.1 | 224 ± 3 | 255 ± 4 | 218 ± 3 | 176 ± 4 |
| 0.15 | 259 ± 8 | 300 ± 7 | 239 ± 3 | 168 ± 4 |
| 0.2 | 271 ± 4 | 267 ± 4 | 250 ± 2 | 185 ± 6 |
| 0.25 | 250 ± 3 | 313 ± 4 | 191 ± 8 | 180 ± 2 |
| 0.3 | 246 ± 4 | 290 ± 4 | 206 ± 11 | 194 ± 5 |
| 0.4 | 244 ± 6 | 272 ± 5 | 205 ± 6 | 200 ± 5 |
| 0.5 | 265 ± 4 | 300 ± 4 | 137 ± 6 | 209 ± 5 |

TABLE 2
PDI of the formulations

| | Lipid-10 | Lipid-11 | Lipid-12 | Lipid-13 |
|---|---|---|---|---|
| R_{MO} | (OAPy-4) | (OAPy-2) | (OAMo-1) | (OAPi-1) |
| 0.05 | 0.16 ± 0.03 | 0.23 ± 0.04 | 0.23 ± 0.03 | 0.25 ± 0.03 |
| 0.1 | 0.21 ± 0.02 | 0.18 ± 0.03 | 0.20 ± 0.05 | 0.20 ± 0.04 |
| 0.15 | 0.2 ± 0.05 | 0.20 ± 0.03 | 0.20 ± 0.05 | 0.23 ± 0.04 |
| 0.2 | 0.19 ± 0.03 | 0.23 ± 0.02 | 0.30 ± 0.03 | 0.22 ± 0.05 |
| 0.25 | 0.39 ± 0.03 | 0.28 ± 0.02 | 0.25 ± 0.03 | 0.19 ± 0.03 |
| 0.3 | 0.22 ± 0.02 | 0.20 ± 0.02 | 0.25 ± 0.05 | 0.23 ± 0.03 |
| 0.4 | 0.24 ± 0.03 | 0.10 ± 0.02 | 0.40 ± 0.04 | 0.15 ± 0.05 |
| 0.5 | 0.25 ± 0.04 | 0.21 ± 0.03 | 0.38 ± 0.03 | 0.18 ± 0.05 |

Tables 1 and 2 show the particle size and polydispersity index (PDI) data for nanoformulations prepared by adding amino lipids to MO dispersed with stabiliser Pluronic F-127. The amount of amino lipid added to the formulation is represented by R_{MO}, which is defined as the wt/wt ratio of amino lipid to total lipid, and may be referred to as RAL herein. Measurements were averaged from triplicate, and the results were reported as a Mean+/−standard deviation.

In general, the nanoparticles had a hydrodynamic diameter ranging from 137 nm to 300 nm, and the PDI was in a range from 0.15-0.40 (Table 1 and Table 2). To evaluate the stability of the nanoparticles at RT, the nanoparticles' particle size was also measured 30 days of standing at RT after preparation. No significant difference in size or physical appearance was observed, indicating that the nanoparticles were stable for at least 30 days at room temperature (data not shown).

### Example 4: Phase Behaviour of LLC Lipid Nanoparticles

Effects of amino lipid concentration and pH on the mesophase of the nanoparticles were determined using high throughput formulation and synchrotron small angle X-ray scattering (SAXS).

Nanoparticle preparations (50 μL) were loaded into a transparent 96-well half-area polystyrene plate (Greiner Bio-One, Interpath Inc., VIC, Australia), and 50 μL of a suitable buffer solution (Citrate buffer) of specified pH was added to the plate. Phase behaviour of the nanoparticles was characterized at the SAXS/WAXS beamline of the Australian Synchrotron. The instrument used an X-ray wavelength of λ=1.128 Å (11.0 keV) with atypical flux of approximately 10 photons/s. The sample to detector distance was chosen as 1.6 m which provided a q-range of 0.01-0.5 Å⁻¹ (scattering vector q=4π sin (θ/2)/λ, whereby θ is the scattering angle and λ is the wavelength). Sample scattering patterns were obtained at 25° C. and 37° C. following the procedure reported in the literature. The plate was placed in a holder perpendicular to the X-ray beam. The sample holder was moved by motors that were controlled by in-house ScatterBrain software for automatic SAXS screening. The sample temperature was controlled by a circulating water bath and kept at 25° C. or 37° C. Two-dimensional X-ray diffraction images were recorded on a Decris-Pilatus 1-M detector using ScatterBrain. The scattering images were integrated into one dimensional plots of intensity versus q for phase identification. The mesophase of the nanoparticles were assigned by matching the relative positions of the scattering peaks with known patterns for bicontinuous cubic, hexagonal, lamellar, and micellar cubic phase. The lattice parameter of the nanoparticles were calculated using the IDL-based software AXcess following the described method (Tran et al. 2018; Tran et al. 2016).

### Results

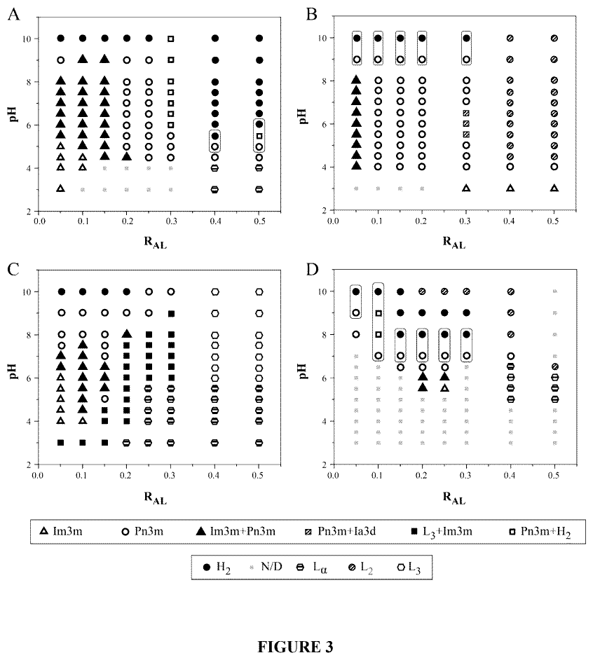

As detailed in Example 2 above, lipid nanoparticles described herein were prepared by doping the synthesised amino lipids into MO at 8 different compositions (R_{MO}=0.05, 0.1, 0.15, 0.2, 0.25, 0.3, 0.4, 0.5) and dispersed with Pluronic F-127. Mesophase structure of the drug-free formulations was determined by SAXS over ten different pH values (a pH range of 2.5 to 10). FIG. 3 represents the SAXS partial phase diagrams of these nanoparticles at 25° C. The shape of an amphiphile can be characterised by the critical packing parameter (CPP). CPP=V/al, in which V is the effective volume of the amphiphile's hydrophobic tail, a is the effective cross-sectional area of the headgroup, and l is the effective length of the hydrocarbon chain. Lamellar phases (L_{α}) are typically observed with a zero interfacial curvature. For CPP values greater than 1, inverse phases, such as Q₂ and H₂ phases, are formed with increasing negative interfacial curvatures. The Q₂ phase consists of lipid bilayers arranged in periodic 3D structures by twisting the bilayers into the shape of infinite periodic minimal surfaces. The H₂ is a closed, extended micellar columnar structure, where there is no direct contact between the water inside and outside the H₂ phase.

The high throughput SAXS experiments demonstrated that both amino lipid composition and pH influenced the internal mesophase of the nanoparticles, as discussed in the following.

### (a) Effect of Adding Amino Lipid to MO Nanoparticles at Neutral pH

In the case of MO, the headgroup size is relatively small, leading to the formation of an inverse bicontinuous cubic phase with a negative surface Gaussian curvature in an aqueous environment. Previous studies showed that MO nanoparticles stabilised with Pluronic F-127 exhibited a primitive cubic phase (Im3m space group) at room temperature (Tran et al. 2015; Sarkar et al. 2018). At neutral pH, for Lipid-10, Lipid-11 and Lipid-13 adding an increasing amount of amino lipid to MO nanoparticles resulted in changes to the mesophase structure following a sequence of bicontinuous cubic→hexagonal→inverse micelles (Q₂→H₂→L_{α}). The sequential change can be rationalised through the changes in the effective molecular critical packing parameter (CPP). At neutral pH, the addition of Lipid-10, Lipid-11 and Lipid-13 to MO could further increase the effective CPP, inducing more curvature in the lipid membrane. MO has 2-OH (hydroxyl) group as part of its head group, and hydroxyl group also can participate in hydrogen bonding with adjacent water molecules, making the effective headgroup area of MO larger than that of Lipid-10, Lipid-11 and Lipid-13.

For nanoparticles doped with Lipid-10, the transition from Q₂ to H₂ at pH 7 occurred at R_{MO}=0.4. For nanoparticles doped with Lipid-11 at R_{MO}=0.4, L₂ phase was observed.

Lipid-10 and Lipid-11 prepared herein can be contrasted with comparator lipids Lipid-1 and Lipid-2 having an ester-rather than an amido-linker (Rajesh et al. 2021, the contents of which are incorporated herein by reference). The amino lipids disclosed herein contain an amide group in the linker while the published comparator lipids were ester-containing amino lipids. Interestingly, the transition from Q₂ to H₂ at neutral pH occurs for Lipid-10 doped MO nanoparticles at a much higher R_{MO} compared to Lipid-1. The fact that the transition from Q₂ to H₂ at pH 7 occurs for the present amide-containing amino lipids at a much higher R_{MO} is most likely due to the hydrophilicity of the amide bond. The partition coefficient P is defined as the ratio of the concentration of a compound between organic and aqueous phases, and the log P value can be employed as a measure of lipophilicity. Predicted log P values for comparator Lipid-1 and the presently disclosed Lipid-10 are 6.95 and 6.12 (Chemdraw software), respectively, indicating that the comparator amino lipid Lipid-1 is more lipophilic.

For Lipid-12, the opposite trend was observed, such that adding an increased amount of Lipid-12, the change was from bicontinuous cubic mesophase→(mixed cubic+sponge) mesophase→sponge (Q₂→Q₂+L₃→L₃) mesophase. L₃ phase is usually found in a narrow region between L_{α} and Q₂ phases and represents “melted” cubic phase structure without long-range order. The SAXS diffraction pattern for the L₃ phase contains only a single broad scattering peak, due to the absence of periodic structures. The co-existence of the sponge phase and Q₂ phase was observed and adding more Lipid-12, results in a single sponge phase. This difference is attributed to the chemical structure of Lipid-12, amide linker attached to hydrophilic morpholine, making the effective headgroup area larger than that of MO. Adding an additive which is more polar or has larger head group size than MO could further decrease the effective CPP, inducing less curvature in the lipid membrane.

### (b) Effect of pH on Mesophase Structures of Amino Lipid-Containing MO Nanoparticles

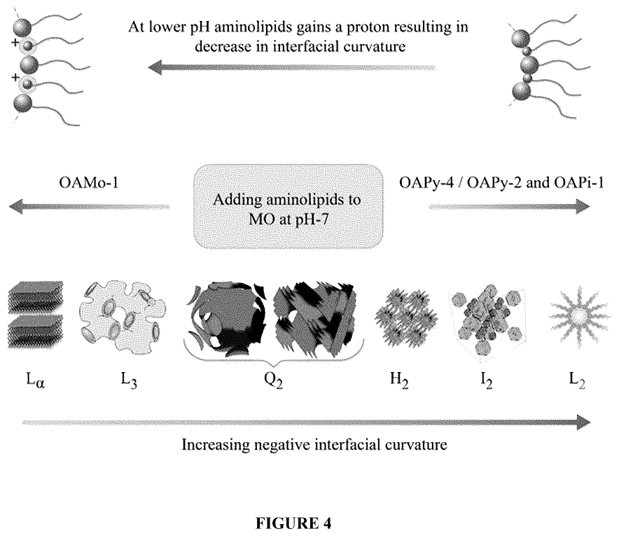

For each system, the pH was varied from pH 2 to pH 8, and Synchrotron SAXS was used to determine the effect of pH on the mesophase structure of MO nanoparticles doped with studied four different amino lipids. SAXS results for nanoparticles stabilised by F-127 are presented in FIG. 3. MO nanoparticles stabilised with F-127 exhibited a primitive cubic phase independent of pH between pH 2.5 and pH 8 (data not shown). In general, as pH decreased, the mesophases of amino lipid-doped MO nanoparticles transformed to structures with lower interfacial curvature. This observation confirmed that the ionisation state of the amino lipid plays a vital role in pH responsiveness of the MO nanoparticles. Headgroups containing tertiary amine moieties gain a proton at low pH and acquire a positive charge resulting from the protonation. This combining with the electrostatic repulsion of the ionised headgroups results in larger effective headgroup size and a smaller CPP. At higher pH, depending upon the apparent pKa of the amino lipid, the headgroup of the amino lipid remains unionised and hence has a small effective headgroup (larger CPP). Therefore, in the self-assembled lipid microenvironment, the decrease of pH leads to the transition from high to low CPP, or in other words a phase transformation sequence of L₂→H₂→Q₂ reducing the interfacial curvature, as summarized in FIG. 4. As represented in FIG. 4, at pH-7 adding Lipid-10, 11 or Lipid-13 the observed phase transition is from Q₂→H₂→L₂, and phase transition from Q₂→Q₂+L₃→L₃ is observed for nanoparticles doped with Lipid-12. Changing pH resulted in a decrease in interfacial curvature, and transition trend followed the sequence L₂→H₂→Q₂.

The ionisable headgroup moiety of the amino lipid resides at the lipid-water interface. Each of the nanoparticle formulations prepared above is unique with types of amino lipid and amount are different in each of them and hence can have different apparent pKa, as the pH and ionic strength are modified. This apparent pKa is primarily influenced by the interfacial solvating medium effect and the surface charge density at the interface for weak bases. The surface charge density at the interface will be governed by the degree of headgroup ionisation (pH effect) and the ionic strength (electrostatic screening effect) (Drummond et al., 1989).

The pH range in which phase transition occurred depended on both the molecular structure of the amino lipid and its doping level in MO nanoparticles. For Lipid-10, the desired phase transition from H₂ at higher pH 7.5 to Q₂ at pH 5.5 was observed at concentration range R_{MO}=0.4-0.5, and a further decrease in pH 4.0 resulted in liposome formation. For Lipid-11 at R_{MO}=0.4-0.5, the transition from L₂ to Q₂ was observed at pH 3.0. For Lipid-12, lowering the pH resulted in mixed L₃ and weekly ordered Q₂ and a further reduction in pH into a L_{α} phase. For Lipid-13, at R_{MO}=0.2-0.3, the desired H₂→Q₂ transition was observed at pH 7.0, the systems appear to be very sensitive, with the change from H₂ to Q₂ observed within 0.5 pH unit difference. It should be noted that for systems with Lipid-13, there are large areas in the phase diagram where the mesophase cannot be determined (denoted N/D). For these samples, the scattering peaks were either too weak or not found precluding phase identification.

The results indicated that the studied ionisable amino lipids promote the formation of a H₂ phase in MO nanoparticles at neutral pH. However, at acidic pH, the amino lipids are gradually protonated, and the electrostatic repulsion among the protonated headgroups at the lipid-water interface reduces the surface curvature. It is believed that as a result, a decrease in pH triggers a phase transition, converting hexosomes into cubosomes at the acidic condition. Out of the exemplary four amino lipid-MO nanoparticle systems, two of them namely Lipid-10 and Lipid-13, displayed a 15 pH-dependent H₂→Q₂ phase transition at pH (pH 5.0-pH 6.5), which is a pathologically relevant range for solid tumours.

### (c) Effect of Temperature on Mesophase Structure of Amino Lipid-Containing MO Nanoparticles

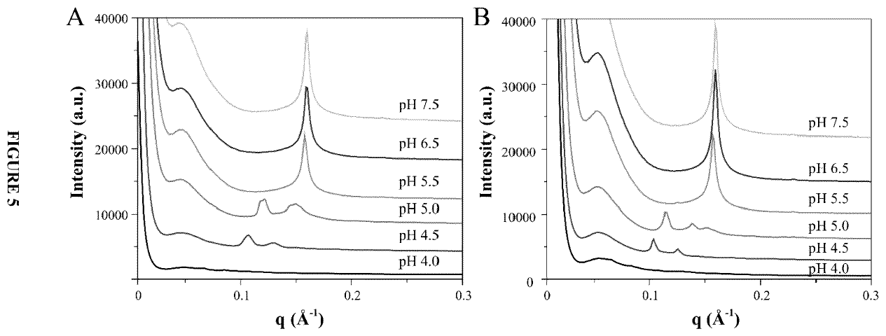

Lyotropic liquid crystalline phases are temperature dependant and hence to evaluate the fate of nanoparticle at body temperature, SAXS experiments were also conducted at 37° C. to study and compare the phase behaviour of the nanoparticles at 25° C. and 37° C. For this purpose selected the formulation with Lipid-10 at R_{MO} 0.4, which exhibited H₂ at pH 7.5 and transitioned to Q₂ at lower pH. One-dimensional SAXS profile for the nanoparticles at pH 4.0 to pH 7.5 are shown in FIG. 5. From the SAXS profile, it is evident that temperature does have a slight effect on the mesophase structure of amino lipid doped nanoparticles. At 37° C., the phase transition from H₂ to Q₂ by 0.5 pH unit higher compared to that at 25° C. Nevertheless, both systems were eligible for loading with an active agent using the drug SN-38, and were investigated further for drug loading, encapsulation efficiency (EE) and drug release studies.

### Example 5: Preparation of LLC Lipid Nanoparticles Comprising an Active Agent

The LLC lipid carrier disclosed herein, such as LLC lipid nanoparticles disclosed, herein may comprise an active agent, such as a chemotherapy drug, cosmetically active molecule, or a nucleic acid. In the following examples, potent anticancer drug SN-38 was used as the active agent.

Drug-comprising formulations were prepared by first preparing the dried lipid mixture of MO and amino lipid described in Example 2 above. Solid SN-38 was added to the dried lipid mixture (20 mg). The resulting mixture was heated at 60° C. overnight. The resulting mixture was mixed using a vortex mixer for 2 min and then an aqueous solution of Pluronic F-127 (2 mg/ml) was added. The ensuing mixture was probe sonicated in pulse mode for 5 min (Ranneh et al., 2016).

### Results

In this example, 1, 2, 5 or 10 wt % of SN-38, to the total amount of lipid mixture in the formulation, was added to pH-responsive MO nanoparticles containing Lipid-10 (R_{MO}=0.4) to prepare four formulations with different quantity of SN-38.

Visual observation of the formed formulations suggested that the formulations were dispersed well, although some precipitate was observed, specifically with 5 wt % and 10 wt %.

### Example 6: Particle Size and Polydispersity Index of the LLC Lipid Nanoparticles Comprising an Active Agent

The SN-38 containing lipid nanoparticles prepared as described in Example 5 above were studied for their physiochemical characteristics.

### Results

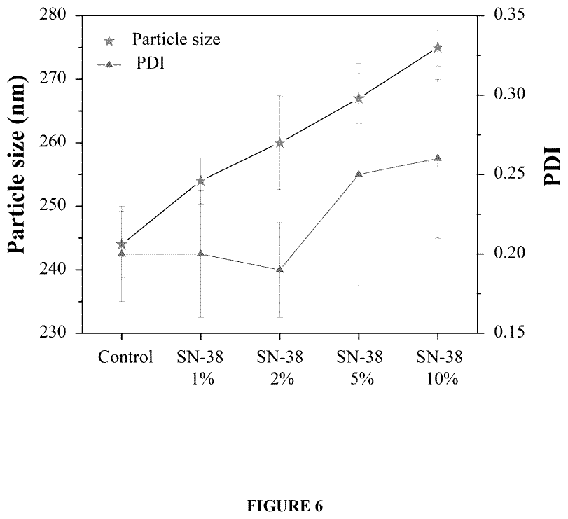

Each of the four lipid nanoparticle formulations was investigated for its particle size and polydispersity index (PDI) as described above in Example 3, and the results are shown in FIG. 6.

The particle size of the drug-loaded formulation was in a range of 244-274 nm with PDI 0.19-0.26. In general, there was a slight gradual increase in the particle size as more SN-38 was added into the system. Carsado et al., reported the lipid/drug molar ratio strongly influenced the size and the amount of SN-38 inside vesicles and observed a significant increase in liposomal size for their novel microfluidic liposomal formulation for delivery of SN-38 (Carsado et al., 2018). However, in formulations with Lipid-10, the average particle size increase was around 10-15%, and these are suitable for delivering SN-38.

### Example 7: Phase Behaviour of the LLC Lipid Nanoparticles Comprising an Active Agent

Mesophase structure of each of the SN-38 containing lipid nanoparticle formulations prepared as described in Example 5 was determined at a pH range.

### Results

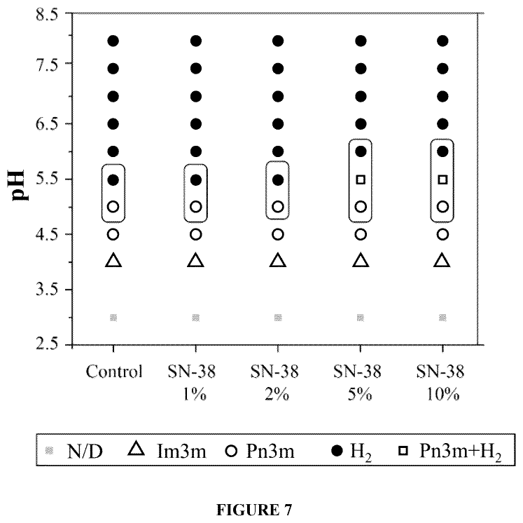

SAXS experiments were conducted, as described above in Example 4, to study internal mesophase structure in response to a pH in the range of 2.5 to 8 at 25° C. (data not shown) and at 37° C. Partial phase diagram at 37° C. is represented in FIG. 7 and compared with the formulation without the drug.

SAXS experiments were conducted with the drug-loaded formulations to see the effect of drug loading on the mesophase behaviour and compared them to the formulation without the drug. It was interesting to see all the drug-loaded formulations behaved slightly different than the control without the drug. For formulations with 1, 2 and 5 wt %, the phase transition from H₂→Q₂ occurred at pH 5.0. For 10 wt % ratio drug loaded formulation co-existing phase window of (X+H₂) was observed at pH 5.5, where X is not an identifiable phase and probably a weakly scattering Q₂ phase. Complete transition to a Q₂ phase occurred at pH 5.0. Slightly higher H₂→Q₂ transition pH can be explained by studied conducted by Casado et al., for interaction between SN-38 drug and biomembrane models and reported that SN-38, inserted within the hydrophobic core of the bilayers due to their high hydrophobicity nature and drug localisation in the outer hydrophobic zone of the bilayer (Carsado et al., 2018). H₂→Q₂ phase transfer pH moving up by 0.5 pH unit will be an added advantage and results are encouraging as SN-38 can be inserted within the hydrophobic core of a mixed lipid bilayer, without disturbing its structure or affecting the stability of the nanoparticles.

SAXS results demonstrated that drug loading did not alter the mesophases structure at neutral pH and drug loading shifts the phase transition (H₂→Q₂) pH to 0.5 unit higher than the control without the drug.

### Example 8: Drug Loading and Drug Encapsulation Efficiency of the LLC Lipid Nanoparticles Comprising an Active Agent

The amount of drug loading (DL) and encapsulation efficiency (EE) were determined for each of the four formulations of Example 5 using an HPLC method.

The nanoparticles containing the drug was centrifuged at 1000×g for 25 min at 25° C. to remove any un-encapsulated drug agglomerates. The supernatant was collected and vortexed for 10 min at 2500 rpm for uniform distribution.

For the SN-38 drug content assay, a UV variable detector at a wavelength of 265 nm and an Agilent Zorbax SB-C18 column (4.6 mm×250 mm, five μm) were utilised. The mobile phase consisted of a 50:50 (v/v) mixture of 25 mM NaH₂PO₄ (pH=3.1) buffer and acetonitrile. The mobile phase pH was maintained at 3.8 to ensure the analyte, SN-38, was in the closed lactone ring form during the assay. The sample for HPLC was prepared by diluting the nanoparticles 100 times with HPLC solvents (10 μL of the formulation with 990 μL 50/50 mixture of buffer and acetonitrile). During the assay, an aliquot of 20 μL of the sample was injected in duplicate into the HPLC system at a flow rate of 1 ml/min. The SN-38 content in the formulation was quantitatively determined using a standard curve prepared as outlined in previously published journal articles (Zhang et al. 2004; Escoriaza et al. 2000).

The following equation was used calculate the encapsulation efficiency (EE %)

$$E E \% = \frac{Wa}{Wi} \times 100$$

Wa is the weight of SN-38 after centrifugation, and Wi is the weight before centrifugation.

Formulations were prepared by adding 1, 2, 5 and 10 wt % of SN-38 to the total amount of lipid mixture in the formulation. The non-encapsulated free drug was removed from the formulation using a centrifugation method, and % EE was determined using HPLC, as outlined above. When 1 wt % of the drug was added to the formulation, an average % EE of 82% was found, indicating most of the added drug was encapsulated in the bilayer, and there was only a tiny quantity of the free non-encapsulated drug. For drug loading with OAPy-4, it was observed that the higher the amount of drug to lipid ratio, the more % EE was lowered. Each drug has specific requirements for efficiently and stably encapsulated into the lipid nanoparticles.

### Results

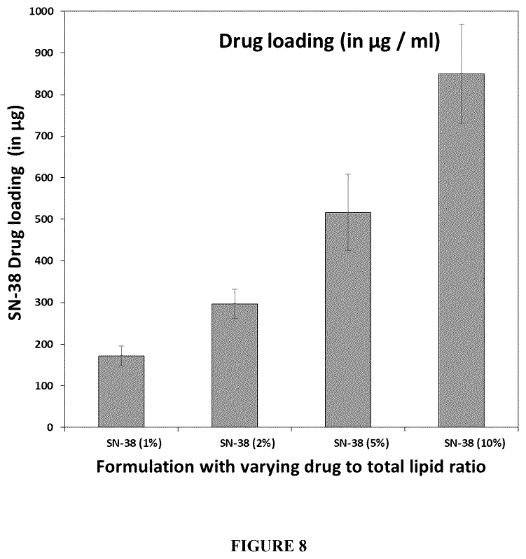

DL and EE % for SN-38 loaded formulations are shown in Table 3 and FIG. 8. For the Table 3 data, representing drug loading and % EE for samples prepared by adding 1, 2, 5 and 10 wt % of SN-38 to the total quantity of lipid mixture in the formulation (MO+OAPy-4) and dispersed with F-127, experiments were performed in triplicate on three independently prepared SN-38 loaded formulations, and all values are expressed as mean+SD (n=3).

TABLE 3

| | Entrapment efficiency | Drug loading |
|---|---|---|
| Formulation | (% EE) | (in μg) |
| SN-38 (1%) | 82 ± 13 | 172 ± 24 |
| SN-38 (2%) | 74 ± 11 | 297 ± 35 |
| SN-38 (5%) | 51 ± 18 | 516 ± 92 |
| SN-38 (10%) | 42 ± 14 | 844 ± 119 |

High loading of 844 μg/ml of SN-38 was achieved, which is ˜100 times greater than the solubility of SN-38 in water. A prior study conducted by Ranneh et al. loaded the SN-38 in a phytantriol-based cubosomes with surfactants to enhance the solubility and using cationic surfactants achieved a high loading formulation of SN-38 with the loading of 90-120 μg/ml (Ranneh et al., 2016). Compared to study conducted by Ranneh, the present formulations with ionisable amino lipids are ˜8 times more loaded.

Dynamic properties of the bilayer could greatly hinder or facilitate the incorporation of hydrophobic molecules such as SN-38. Although carriers with rigid bilayers have evident advantages such as low permeability and stability in vivo, they suffer from many drug loading restrictions. In contrast, fluid bilayers more easily accommodate hydrophobic molecules inside their hydrophobic core. For drug loading with Lipid-10, it was observed that higher the amount of drug to lipid ratio, % EE was lowered. Each drug has specific requirements to be efficiently and stably encapsulated into the nanoparticles. Many factors contribute to the final formulation's success among these, the lipid composition and the manufacturing process that determine the size, surface charge, and bilayer fluidity (Bala et al., 2013). It was observed that loading is pH dependant and nanoparticles prepared with PBS buffer had better loading prepared with DI water. Higher loading with PBS maybe because, with the increase in pH, SN-38 undergoes a structural change from a closed lactone ring to an open carboxylate form, and at pH 6.7 both forms are in equilibrium. Open carboxylate form is water-soluble and hence the incorporation into the water channels along with bilayer and higher drug loading. The efficiency of drug loading will depend on its encapsulation in the aqueous core, its incorporation into the bilayer, or its partition between these two phases.

Overall, the results indicate that the novel LLC lipid nanoparticles disclosed herein enable a high drug loading of the drug SN-38.

### Example 9: Drug Release Study

In vitro release of SN-38 from the nanoparticles was evaluated using a dynamic dialysis method (Zambito, Y. International Journal of Pharmaceutics 2012, 434 (1), 28-34). The drug release rate was determined by placing the dispersion in a dialysis tube (Pur-A-Lyzer™ Maxi Dialysis tube molecular weight cut off 3500) and exhaustively dialysing.

After removing the free drug, the highest SN-38 loaded nanoparticles (1 mL formulation) were placed inside the dialysis tube. The dialysis tube was then immersed in a 1000 mL buffer maintained at 37±1.0° C. and stirred at 400 rpm. The analysis of the separation of free drug from drug-loaded formulations was carried out over a period of 420 min. Release experiments were performed in triplicate on three independently prepared SN-38 loaded formulations. For physiological pH, experiments were carried out by dialysing the formulations against 1000 mL of phosphate buffer (pH 7.0). Lower pH experiments were carried out by dialysing the formulations against 1000 mL of phosphate buffer adjusted to the required pH. The SN-38 that remained in the dialysis tube was quantitatively measured by the HPLC method. At predetermined time intervals (60 min, 120 min, 240 min and 420 min), 10 μL of the sample was withdrawn from the dialysis tube and diluted with 990 μL of the HPLC solvent. The protocol for predicting the release kinetics from withdrawing the sample from inside the dialysis bag is described in the literature.

### Results

A calibration experiment of drug diffusion through the dialysis membrane in the absence of lipid nanocarriers drug delivery vehicles was performed to evaluate the effect of the dialysis membrane on the release kinetics. More than 90% of SN-38 is released from a pure-drug suspension within 60-90 min (data not shown here), and therefore any membrane effect is negligible with respect to drug release kinetics.

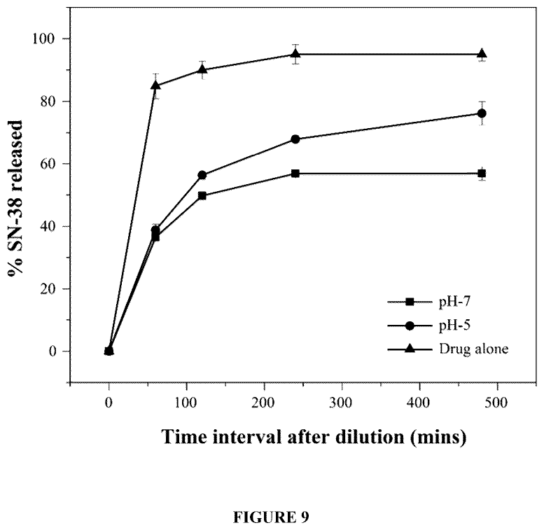

The release profiles of SN-38 from the SN-38-Lipid 10 nanoparticles at different pH values are shown in FIG. 9. The SN-38-Lipid 10 formulations showed burst release (˜37%) for the first 60 min of the study for both studied pH (7.0 and 5.0) and a sustained release after the first hour. In dialysis settings, the sink condition was maintained at 10-20 times higher than the volume required for saturated drug solution so that the free drug could quickly equilibrate across the dialysis membrane. Since the solubility of SN-38 in water is 7-11 μg/mL, we have used 1000 mL of sink solution for 1 mL of the formulation. This 1000 fold dilution increases the diffusion-controlled drug release rate by orders of magnitude, resulting in an apparent burst release of SN-38.

After the first hour of the study, compared to the samples at pH=7.0, the drug release rate from the samples at pH=5.0 was faster. This difference in sustained release behaviour is consistent with the presence of different mesophase (inverse hexagonal or cubic phases) under the different pH conditions. The cubic phase with two interpenetrating water channels, likely open to the external aqueous phase, provides more surface areas and more accessible routes for drug release. On the other hand, the hexagonal phase with closed columnar micelle structures hinders drug release. Drug release from the bicontinuous cubic phase network of large open channels is released more quickly than hexagonally packed channels of smaller diameters.

Previous in vitro and in vivo studies have shown that altering the release rate is one of the possible ways to increase drug distribution to the tumour and improve the anti-tumour effect of the drug. The release results for the SN-38-Lipid 10 nanoparticles reveal a slower drug release rate in the simulated normal cell physiological environment (pH=7.4) and a faster release rate in the simulated tumour microenvironment and lysosome environment (pH=5.0). The slower release rate at pH 7.4 is potentially beneficial, as this may lead to decreased drug loss in circulation and increased bioavailability of a drug at the tumour site. The faster release rate at pH=5.0 is beneficial, as this leads to accumulation of the drug in pathological environments with reduced cytotoxicity of SN-38 for healthy cells and improved efficacy.

The results indicate that the novel LLC lipid nanoparticles disclosed herein enable a controlled release of the drug SN-38 in response to pH.

### Example 10: In Vivo Toxicity Study of the LLC Lipid Nanoparticles

In vivo toxicity of the LLC lipid nanoparticles disclosed herein was evaluated in mice.

Nanoparticles were prepared using evaporation and sonication methods described above using the amino lipids OAPi-1 and OAPy-4, and monoolein (MO) as the structural lipid:

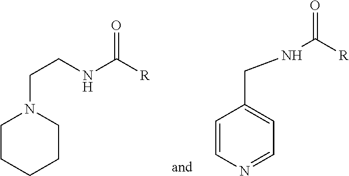

### Structures of OAPi-1 N-(2-(piperidin-1-yl)ethyl)oleamide) and OAPy-4 (N-(pyridine-4-ylmethyl)oleamide)

Briefly, the lipids were dissolved at appropriate ratios in ethanol. The ratio of MO:OAPi-1 was 75:25 and 85:15 (wt:wt). The ratio of MO:OAPy-4 was 60:40. The solvent was evaporated in a vacuum oven at 40° C. overnight. Aqueous solutions containing Pluronic F-127 was added to the dried lipid mixtures. The ratio of F-127 to lipid was kept a constant at 1:10 (wt/wt). The solution was sonicated using a probe sonicator to produce stable nanoparticles.

Six-weeks old Balb/c nude mice were injected intraperitoneally with the formulated nanoparticles at specified doses (50, 100, and 200 mg/kg). The mice were monitored over 24 hours for any adverse effects.

| | | MO - OAPi-1 | MO - OAPi-1 | |
|---|---|---|---|---|
| | | 75:25 | 85:15 | |
| | | nanoparticles | nanoparticles | MO - OAPy-4 |
| Concentration* | | (Formulation 1) | (Formulation 2) | nanoparticles |
| 50 | mg/kg | No observable | No observable | No observable |
| | | effects after | effects after | effects after |
| | | injection and after | injection and after | injection and after |
| | | 24 hr | 24 hr | 24 hr |
| 100 | mg/kg | Same | Same | Same |
| | | observations | observations | observations |
| 200 | mg/kg | Same | Same | Same |
| | | observations | observations | observations |

*Note:
concentration here refers to the lipid concentration in the formulation.

The results showed that at the tested concentrations, the nanoparticles showed no adverse effects after 24 hours. This experiment suggests that the nanoparticles do not cause acute toxicity in mice. It also suggests that the maximum tolerable dose is higher than 200 mg/kg.

The results indicate that the novel LLC lipid nanoparticles disclosed herein are suitable for clinical applications as a pharmaceutical or cosmetic agent carrier.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### REFERENCES

Bala, V.; Rao, S.; Boyd, B. J.; Prestidge, C. A., Prodrug and nanomedicine approaches for the delivery of the camptothecin analogue SN38. J Control Release 2013, 172 (1), 48-61.
Casadó, A.; Sagristá, M. L.; Mora, M., A novel microfluidic liposomal formulation for the delivery of the SN-38 camptothecin: characterisation and in vitro assessment of its cytotoxic effect on two tumor cell lines. Int J Nanomedicine 2018, 13, 5301-5320.
Drummond, C. J.; Grieser, T. F.; Healy, T. W., Acid-Base Equilibria in Aqueous Micellar Solutions. Journal of the Chemical Society, Faraday Transactions 1. 1989, 85 (3), 521-535.
Escoriaza, J.; Castellanos, A. A. C.; Calvo, E.; Giraldez, J. Simple and rapid determination of Irinotecan and its metabolite SN-38 in plasma by high-performance liquid-chromatography: application to clinical pharmacokinetic studies. Journal of Chromatography B, 2000, 740 (2000) 159-168.
Rajesh, S.; Zhai J.; Drummond, C. J., Tran, N. Synthetic ionizable aminolipids induce a pH dependent inverse hexagonal to bicontinuous cubic lyotropic liquid crystalline phase transition in monoolein nanoparticles. Journal of Colloid and Interface Science, 2021, Volume 589, 85-95.
Ranneh, A. H.; Iwao, Y.; Noguchi, S.; Oka, T.; Itai, S. The use of surfactants to enhance the solubility and stability of the water-insoluble anticancer drug SN38 into liquid crystalline phase nanoparticles. Int J Pharm 2016, 515 (1-2), 501-505.
Sarkar, S.; Tran, N.; Rashid, M. H.; Le, T. C.; Yarovsky, I.; Conn, C. E.; Drummond, C. J. Toward Cell Membrane Biomimetic Lipidic Cubic Phases: A High-Throughput Exploration of Lipid Compositional Space. ACS Applied Bio Materials 2018, 2 (1), 182-195.
Tran, N.; Hawley, A. M.; Zhai, J.; Muir, B. W.; Fong, C.; Drummond, C. J.; Mulet, X., High-Throughput Screening of Saturated Fatty Acid Influence on Nanostructure of Lyotropic Liquid Crystalline Lipid Nanoparticles. Langmuir 2016, 32 (18), 4509-4520.
Tran, N.; Hocquet, M.; Eon, B.; Parveen Sangwan; Ratcliffe, J.; Hinton, T. M.; White, J.; Ozcelik, B.; Reynolds, N. P.; Muir, B. W., Non-Lamellar Lyotropic Liquid Crystalline Nanoparticles Enhance the Antibacterial Effects of Rifampicin against Staphylococcus Aureus. Journal of colloidal and interface Science 2018, 519 (1), 107-118.
Tran, N.; Mulet, X.; Hawley, A. M.; Hinton, T. M.; Mudie, S. T.; Muir, B. W.; Giakoumatos, E. C.; Waddington, L. J.; Kirbyb, N. M.; Drummond, C. J., Nanostructure and cytotoxicity of self-assembled monoolein-capric acid lyotropic liquid crystalline nanoparticles. Royal Society of Chemistry Advances 2015, 5 (34), 26785-26795.
Zhai, J.; Hinton, T. M.; Waddington, L. J.; Fong, C.; Tran, N.; Mulet, X.; Drummond, C. J.; Muir, B. W., Lipid-PEG conjugates sterically stabilise and reduce the toxicity of phytantriol-based lyotropic liquid crystalline nanoparticles. Langmuir 2015, 31 (39), 10871-10880.
Zhang, J. A.; Xuan, T.; Parmar, M.; Ma, L.; Ugwu, S.; Ali, S.; Ahmad, I., Development and characterisation of a novel liposome-based formulation of SN-38. Int J Pharm 2004, 270 (1-2), 93-107.

1. A lyotropic liquid crystalline (LLC) lipid carrier comprising a structural lipid which is a lyotropic liquid crystal phase forming lipid, and an amino lipid having an amido-linker, the LLC lipid carrier adapted to undergo a mesophase transition upon exposure to a drop in pH.

2. The LLC lipid carrier of claim 1, wherein the structural lipid is capable of forming a cubic mesophase structure.

3. The LLC lipid carrier of claim or claim 2, wherein the structural lipid comprises a hydrophobic tail group selected from the group consisting of oleyl, linoleoyl, linolenoyl, phytanoyl, famesoyl or extended aliphatic hydrophobic, optionally selected from oleyl, linoleoyl, and phytanoyl.

4. The LLC lipid carrier of any one of claim 1 to claim 3, wherein the structural lipid is monoolein (glycerol monooleate).

5. The LLC lipid carrier of any one of claim 1 to claim 4, wherein the amino lipid is present at a suitable wt % of the total lipid content of the LLC lipid carrier to provide for the mesophase transition upon exposure to a drop in pH.

6. The LLC lipid carrier of any one of claim 1 to claim 5, wherein the amino lipid comprises about 5% to about 50 wt % of the total lipid content of the LLC lipid carrier.

7. The LLC lipid carrier of any one of claim 1 to claim 6, wherein the amino lipid has the structure of Formula (I):
Cyc-L-R (I),
wherein Cyc is a nitrogen heterocycle or heteroaryl;
L is an amido-linker; and
R is a C10 to C44 carbon chain.

8. The LLC lipid carrier of any one of claim 1 to claim 7, wherein the amino lipid has the structure of Formula (Ib): 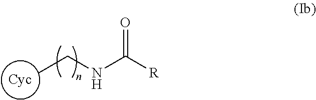
wherein Cyc is a 5- or 6-membered nitrogen heterocyclyl or heteroaryl, optionally selected from the group consisting of pyridyl, pyrimidinyl, piperidinyl, piperazinyl, morpholinyl, and pyrazinyl;
L is an amido-linker;
R is a C10 to C44 carbon chain, optionally a C12 to C24 alkyl or alkenyl, optionally interrupted by one or more heteroatoms; and
n is an integer from 1 and 6.

9. The LLC lipid carrier of claim 7 or claim 8, wherein R is selected from the group consisting of oleyl, linoleoyl, linolenoyl, phytanoyl, and farnesoyl.

10. The LLC lipid carrier of any one of claim 1 to claim 9, wherein the amino lipid is selected from the group consisting of: 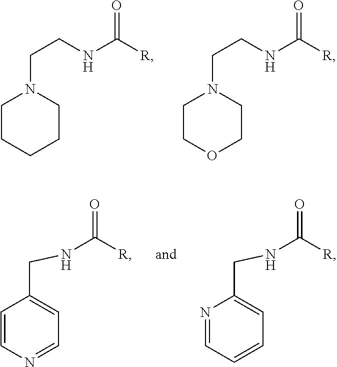
wherein, R is as defined in any one of claim 10 to claim 12.

11. The LLC lipid carrier of any one of claim 1 to claim 10, wherein the LLC lipid carrier further comprises a stabilizer, optionally wherein the stabilizer is a stabilizing polymer, optionally a non-ionic triblock copolymer.

12. The LLC lipid carrier of claim 11, wherein the stabilizer is present in an amount of about 10 wt % of the total lipid content of the LLC lipid carrier.

13. The LLC lipid carrier of claim 12 or claim 13, wherein the stabilizer is Pluronic F127 (Poloxamer 407) or Poloxamer 80.

14. The LLC lipid carrier of any one of claim 1 to claim 13, wherein the LLC lipid carrier is in the form of lyotropic liquid crystalline (LLC) nanoparticles.

15. The LLC lipid carrier of any one of claim 1 to claim 14, wherein the mesophase transition upon exposure to a drop in pH is a transition from a hexagonal phase to a cubic phase.

16. The LLC lipid carrier of claim 15, wherein the LLC lipid carrier comprises a hexagonal lyotropic liquid crystalline phase structure at a pH of about pH 7 or higher, optionally about pH 7.4 or higher.

17. The LLC lipid carrier of claim 15 or claim 16, wherein the LLC lipid carrier comprises a cubic lyotropic liquid crystalline phase structure at a pH of about pH 4.0-pH 7.0.

18. The LLC lipid carrier of any one of claim 15 to claim 17, wherein the LLC lipid carrier comprises a hexagonal lyotropic liquid crystalline phase structure at a pH of about pH 7 or higher, optionally about pH 7.4 or higher, and a cubic lyotropic liquid crystalline phase structure at a pH of about pH 4.0-pH 7.0.

19. The LLC lipid carrier of any one of claim 1 to claim 18, further comprising an active agent, optionally wherein the active agent is pharmaceutically or cosmetically active.

20. The LLC lipid carrier of claim 19, wherein the active agent is selected from the group consisting of peptides, proteins, enzymes, small molecule drugs, and nucleic acids, optionally wherein the active agent is selected from the group consisting of radionuclides, imaging agents, polymers, antibiotics, fungicides, metal-containing nanoparticles, anti-inflammatory agents, anti-tumor agents, cardiovascular agents, anti-anxiety agents, hormones, growth factors, steroidal agents, gene expression modifiers, knockdown agents, siRNA, RNAi agents, mRNA, DNA, dicer substrates, miRNA, shRNA, antisense oligonucleotides, aptamers, and microbially derived toxins.

21. The LLC lipid carrier of claim 19 or claim 20, wherein the active agent is a topoisomerase I inhibitor, optionally selected from camptothecin, irinotecan, and SN-38.

22. The LLC lipid carrier of any one of claim 1 to claim 21, wherein the LLC lipid carrier has a water content of between about 20 to about 60% by weight.

23. A lyotropic liquid crystalline (LLC) lipid nanoparticle composition which is a dispersion of the LLC lipid carrier of any one of claim 19 to claim 22 in a polar medium.

24. A cosmetic composition comprising the LLC lipid carrier of any one of claim 19 to claim 22, or the LLC lipid nanoparticle composition of claim 23, and a cosmetically acceptable carrier, diluent and/or excipient.

25. A pharmaceutical composition comprising the LLC lipid carrier of any one of claim 19 to claim 22, or the LLC lipid nanoparticle composition of claim 23, and a pharmaceutically acceptable carrier, diluent and/or excipient.

26. The cosmetic composition of claim 24, or the pharmaceutical composition of claim 25, wherein the composition is formulated as a composition for injection, for topical administration, or for subcutaneous administration.

27. A method of delivering an active agent to a biological system including the step of administering to the biological system the LLC lipid carrier of any one of claim 19 to claim 22, the LLC lipid nanoparticle composition of claim 23, or the composition of any one of claim 24 to claim 26.

28. The method of claim 27, wherein the delivery of the active agent in the biological system is modified by a response of the LLC lipid carrier to a predefined pH range.

29. The method of claim 28, wherein the response comprises a mesophase structure transition of the LLC lipid carrier, optionally wherein the phase transition is a transition from a hexagonal phase structure to a cubic phase structure of the LLC lipid carrier.

30. The method of claim 29, wherein the mesophase structure transition occurs at a pH of about pH 7.0-pH 4.0, optionally about pH 7.0-pH 5.5.

31. The method according to any one of claim 27 to claim 30, wherein the response provides for a preferential release of the active agent at the predefined pH range in the biological system.

32. A method of controlled release of an active agent comprising the steps of forming the LLC lipid carrier of any one of claim 19 to claim 22, the LLC lipid nanoparticle composition of claim 23, or the composition of any one of claim 24 to claim 26, and administering the LLC lipid carrier, the LLC lipid nanoparticle composition, or the composition to a biological system comprising a target area having a predefined pH to thereby achieve a preferential release of the active agent at the target area.

33. The method of claim 32, wherein the LLC lipid carrier undergoes a mesophase structure transition in response to the predefined pH.

34. The method of claim 33, wherein the mesophase structure transition is a transition from a hexagonal phase structure to a cubic phase structure of the LLC lipid carrier.

35. A method of treatment or prevention of a disease, disorder or condition in a mammal, comprising administering to the mammal a therapeutically effective amount of the LLC lipid carrier of any one of claim 19 to claim 22, the LLC lipid nanoparticle composition of claim 23, or the composition of any one of claim 24 to claim 26.

36. A method of diagnosing a disease, disorder or condition in a mammal including the step of administering the LLC lipid carrier of any one of claim 19 to claim 22, the LLC lipid nanoparticle composition of claim 23, or the composition of any one of claim 24 to claim 26, wherein the active agent is a labelled active agent, to the mammal or to a biological sample obtained from the mammal to facilitate diagnosis of the disease, disorder or condition in the mammal.

37. The method of claim 35 or claim 36, wherein the disease, disorder or condition is a cancer or a bacterial or fungal infection.

38. An amino lipid of Formula (Ib): 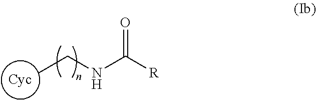
wherein Cyc is a 5- or 6-membered nitrogen heterocyclyl or heteroaryl, selected from the group consisting of piperidinyl, piperazinyl, pyrrolinyl, pyrrolidinyl, pyrazolidinyl, imidazolinyl, imidazolidinyl, pyrrolinyl, pyrazolinyl, thiazolidinyl, thiomorpholinyl, tetrahydropyridyl, dihydropyridyl, tetrahydropyrinidinyl, pyrimidinyl, pyridyl, pyrrolyl, imidazolyl, pyrazolyl, oxazolyl, oxadiazolyl, pyrazinyl, tetrazolyl, thiazolyl, isoxazolyl, isothiazolyl, isoxazolonyl, triazolyl, oxadiazolyl, thiadiazolyl, and pyridazinyl;
L is an amido-linker;
R is a C10 to C44 carbon chain, optionally a C12 to C24 alkyl or alkenyl, optionally interrupted by one or more heteroatoms; and
n is an integer from 1 and 6.

39. The amino lipid of claim 38, wherein Cyc is selected from the group consisting of pyridyl, pyrimidinyl, piperidinyl, piperazinyl and pyrazinyl.

40. The amino lipid of claim 38 or claim 39, wherein R is selected from the group consisting of oleyl, linoleoyl, linolenoyl, phytanoyl, and farnesoyl.

41. The amino lipid of any one of claim 38 to claim 40, wherein the amino lipid is selected from the group consisting of: 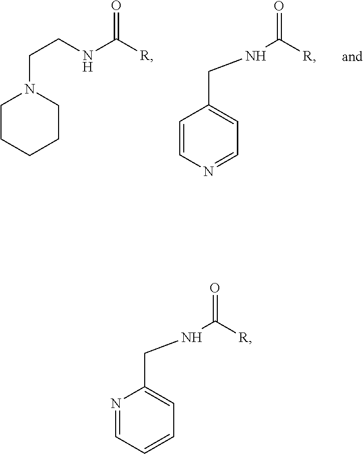
wherein, R is as defined in any one of claim 40 to claim 42.
